(19) United States
(12) Patent Application Publication
(10) Pub. No.: US 2026/0283682 A1
(43) Pub. Date: Sep. 24, 2026

(54) SEAL QUALITY PREDICTION FROM ADVENTITIA FUSING ENERGY

(71) Applicant: CILAG GMBH INTERNATIONAL, Zug (CH)

(72) Inventors: Nicholas A. MCDONOUGH, West Harrison, IN (US); Christopher WAID, Blue Ash, OH (US); Terry MCCOY, Cincinnati, OH (US)

(73) Assignee: CILAG GMBH INTERNATIONAL, Zug (CH)

(21) Appl. No.: 19/567,921

(22) Filed: Mar. 16, 2026

Related U.S. Application Data
(60) Provisional application No. 63/774,372, filed on Mar. 19, 2025.
(60) Provisional application No. 63/851,121, filed on Jul. 25, 2025.
(60) Provisional application No. 63/875,047, filed on Sep. 3, 2025.

(51) Int. Cl. A61B 18/14; A61B 17/00; A61B 18/00

(52) U.S. Cl. CPC A61B 18/1445; A61B 2017/00075; A61B 2018/0063; A61B 2018/00761; A61B 2018/00779; A61B 2018/00791; A61B 2018/00875; A61B 2018/00898

(57) ABSTRACT

A surgical system is disclosed that includes a surgical instrument and a controller in operable communication with the surgical instrument. The surgical instrument includes an end effector that includes a first clamp arm, a second clamp arm movable relative to the first clamp arm from an open configuration to a closed configuration to engage a vessel therebetween, and an electrode. The controller is operable to apply, with the electrode, energy to the vessel, estimate a time of media fracture of the vessel, measure an amount of the energy applied to the vessel after the estimated time, and estimate a quality of a seal applied to the vessel based on the measured amount of the energy.

### CROSS-REFERENCE TO RELATED APPLICATIONS

The present application claims priority under 35 U.S.C. § 119 (e) to U.S. Provisional Application No. 63/774,372, titled “OPTIMIZATION AND COMMUNICATION OF COMPRESSION AND ENERGY FOR SEALING”, filed Mar. 19, 2025, the disclosure of which is hereby incorporated by reference in its entirety.

The present application claims priority under 35 U.S.C. § 119 (e) to U.S. Provisional Application No. 63/851,121, titled “ESTIMATING TISSUE STIFFNESS FROM RINGING”, filed Jul. 25, 2025, the disclosure of which is hereby incorporated by reference in its entirety.

The present application claims priority under 35 U.S.C. § 119 (e) to U.S. Provisional Application No. 63/875,047, titled “SEAL QUALITY PREDICTION FROM ADVENTITIA FUSING ENERGY”, filed Sep. 3, 2025, the disclosure of which is hereby incorporated by reference in its entirety.

### BACKGROUND

The present disclosure relates to surgical instruments and, more particularly, to electrosurgical instruments that are configured to seal and cut tissue.

One type of electrosurgical instrument utilizes radiofrequency (RF) to seal and cut tissue in medical procedures. RF surgical instruments have become widely used due to their ability to provide precise and controlled tissue sealing and cutting. RF surgical instruments utilize electrodes that deliver RF energy to target tissues, causing localized heating that results in cellular destruction and coagulation. The RF surgical instruments may utilize a knife that cuts the heated tissue, such as after cellular destruction or coagulation has completed. These RF instruments have been applied in various surgical fields, including general surgery, dermatology, and minimally invasive procedures.

A typical vessel includes two layers-a tunica media or “media” layer and a tunica adventitia or “adventitia” layer surrounding the media layer. When energy is provided to the vessel with an RF surgical instrument, the media layer typically fractures and the opposing sides of the adventitia layer are fused together, thereby sealing a lumen of the vessel. The sealed vessel may then be cut with a knife of the RF surgical instrument.

In some situations, the vessel may not have been properly and/or completely sealed by the RF surgical instrument and, therefore, transecting the vessel could be potentially hazardous to the patient. Accordingly, systems and methods for increasing a user's knowledge about the status, or quality, of a seal prior to transection of a vessel are desired.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 illustrates an example surgical system that includes a generator usable with an RF surgical instrument, in accordance with at least one aspect of the present disclosure.

FIG. 2 is a schematic block diagram of the surgical system of FIG. 1 where the generator includes an output device, in accordance with at least one aspect of the present disclosure.

FIG. 3 is an example graph that provides gap distance between clamp arms of the RF surgical instrument of FIG. 1 over time, in accordance with at least one aspect of the present disclosure.

FIG. 4A is an example view of the output device of FIG. 2 displaying a graph of gap distance between clamp arms of the RF surgical instrument of FIG. 1 against time, in accordance with at least one aspect of the present disclosure.

FIG. 4B is an example view of the output device of FIG. 2 displaying a first visual indicator indicating that tissue clamped by the RF instrument of FIG. 1 can be sealed and cut, in accordance with at least one aspect of the present disclosure.

FIG. 4C is an example view of the output device of FIG. 2 displaying a second visual indicator indicating that tissue clamped by the RF instrument of FIG. 1 is fragile, in accordance with at least one aspect of the present disclosure.

FIG. 4D is an example view of the output device of FIG. 2 displaying a third visual indicator indicating that tissue clamped by the RF instrument of FIG. 1 is thick, in accordance with at least one aspect of the present disclosure.

FIG. 5A is an example view of the output device of FIG. 2 displaying first and second visual indicators that indicate a stiffness and a thickness, respectively, of tissue captured by the RF surgical instrument of FIG. 1, in accordance with at least one aspect of the present disclosure.

FIG. 5B is the example view of the output device of FIG. 5A including a third visual indicator that indicates the tissue has reached a steady state, in accordance with at least one aspect of the present disclosure.

FIG. 6A is an example view of the output device of FIG. 2 displaying a multiparameter visual indicator that includes an indicator region displayed as a first color and that indicates various parameters of tissue captured by the RF surgical instrument of FIG. 1, in accordance with at least one aspect of the present disclosure.

FIG. 6B is the example view of the output device of FIG. 6B with the indicator region being located within a target region and displayed as a second color different than the first color, in accordance with at least one aspect of the present disclosure.

FIG. 7 illustrates a surgical system that includes a generator usable with an RF surgical instrument, in accordance with at least one aspect of the present disclosure.

FIGS. 8A and 8B provide a schematic flow diagram for controlling the surgical system of FIG. 7, in accordance with at least one aspect of the present disclosure.

FIG. 9 is a graph that includes a signal representative of gap distance between clamp arms of the RF surgical instrument of FIG. 1 over time during use of the RF surgical instrument.

FIG. 10 is an enlarged portion of the graph of FIG. 9.

FIG. 11 is an enlarged portion of the graph of FIG. 9.

FIG. 12 is a schematic flow diagram of an example method for controlling the surgical system of FIG. 1, in accordance with at least one aspect of the present disclosure.

FIG. 13 is a first graph that includes a signal representative of a sensed gap between clamp arms of the RF surgical instrument of FIG. 1 during an amount of time and a regression fit to the signal, in accordance with at least one aspect of the present disclosure.

FIG. 14 is a second graph of an adjusted signal based on the sensed gap and the regression fit FIG. 13, in accordance with at least one aspect of the present disclosure.

FIG. 15 illustrates ringing estimates of various types of tissues, in accordance with at least one aspect of the present disclosure.

FIG. 16 is an example timeline that shows types of energy applied to a vessel with the RF surgical instrument of FIG. 1.

FIG. 17 is a schematic flow diagram of an example method for controlling the surgical system of FIG. 1, in accordance with at least one aspect of the present disclosure.

FIG. 18 is a graph illustrating a gap between clamp arms of the RF surgical instrument of FIG. 1 over time during example uses of the RF surgical instrument while sealing a vessel.

FIG. 19 is a bar graph illustrating weights given to various parameters of a machine learning model for obtaining a status, or quality, of a seal applied to a vessel from the RF surgical instrument of FIG. 1.

### DETAILED DESCRIPTION

Applicant of the present application owns the following U.S. patent applications filed concurrently herewith, the disclosure of each of which is hereby incorporated by reference in their entirety herein:

U.S. patent application Ser. No. 19/567,906, filed Mar. 16, 2026, titled OPTIMIZATION AND COMMUNICATION OF COMPRESSION AND ENERGY FOR SEALING; and

U.S. patent application Ser. No. 19/567,909, filed Mar. 16, 2026, titled ESTIMATING TISSUE STIFFNESS FROM RINGING;

The present disclosure relates to surgical instruments and, more particularly, to radiofrequency (RF) surgical instruments that are configured to seal and cut tissue.

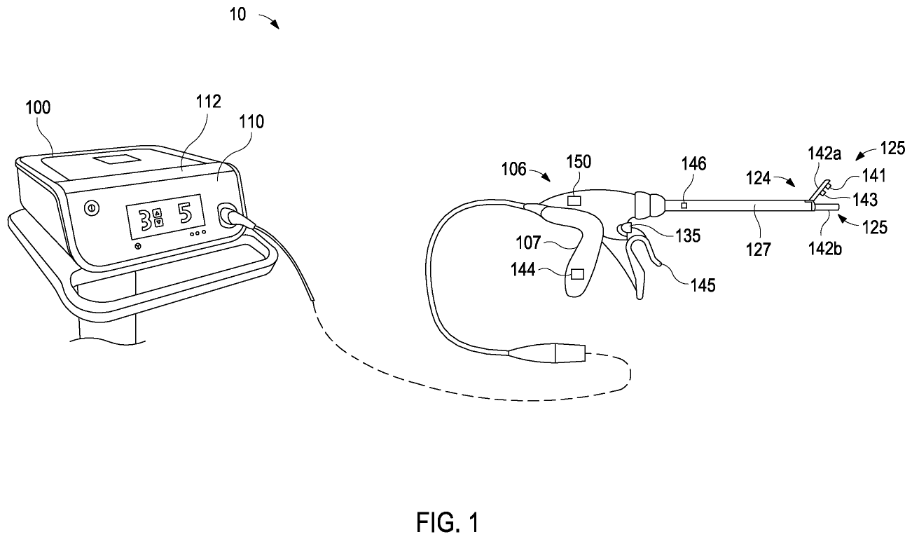

FIG. 1 illustrates an example surgical system 10 comprising a generator 100 usable with an RF surgical instrument 106, in accordance with at least one aspect of the present disclosure. The RF surgical instrument 106 includes a handpiece or “housing” 107, a shaft 127 extending from the housing 107, and an end effector 124 arranged at a distal end of the shaft 127 that includes clamp arms 142a, 142b pivotable relative to one another between an open configuration, as shown in FIG. 1, and a closed configuration (not shown) to grasp tissue therebetween. The clamp arms 142a,b include electrodes 125 that may be energized by a bipolar energy source within the generator 100. The RF surgical instrument 106 may further include a knife (not shown) that can travel through the end effector 124 to cut tissue captured between the clamp jaws 142a,b, such as after the electrodes 125 have applied RF energy to the tissue.

The handpiece 107 comprises a trigger 145 to transition the clamp arms 142,b between the open and closed configurations and an energy button 135 to energize the electrodes 125 in the clamp arms 142a,b. One or both of the clamp arms 142a,b may include a first sensor 141, such as a Hall-Effect sensor, inductive position sensor, capacitive position sensor, an impedance sensor, or an optical sensor, or a combination thereof, to sense a gap distance or angle between the clamp arms 142a,b. The electrodes 125 may also function as sensors, such as impedance sensors, by applying subtherapeutic energy to tissue in contact therewith. Accordingly, the electrodes 125 may be utilized to detect (sense) contact with tissue as the clamp arms 142a,b are transitioned toward the closed configuration. The handpiece 107 may also include a handpiece sensor 144 for measuring a rotational angle of the trigger 145 relative to the handpiece 107, which may be indicative of a gap distance or angle between the clamp arms 142a,b. The shaft 127 may also include a shaft sensor 146 for measuring a position of a component, such as a rotatable drive shaft or a translatable closure shuttle within the shaft 127 used to move the clamp arm 142a between the open and closed position, which may be indicative of a gap distance or angle between the clamp arms 142a,b. The sensors 144, 146 may include an encoder, which may measure a rotational position of the trigger 145 relative to the handpiece 107 or a rotational position of the component within the shaft 127, respectively, which may be indicative of a gap distance or angle between the clamp arms 142a,b.

One or both of the clamp arms 142a,b may further include a second sensor 143, such as a force sensor or a strain gauge, or a combination thereof, to sense a force and/or strain applied by the clamp arms 142a,b to tissue captured therebetween. The electrodes 125 may further operate as a sensor to detect one or more parameters of the tissue captured between the clamp arms 142a,b. For instance, the electrodes 125 may apply a subtherapeutic amount of energy (e.g., an amount of energy not intended to cause a therapeutic effect) to the tissue to detect the impedance of the tissue. The electrodes 125 may be used to detect when the end effector 124 contacts tissue. One or both of the clamp arms 142a,b may further include a third sensor 147, such as a temperature sensor, for sensing a temperature of tissue positioned between the clamp arms 142a,b.

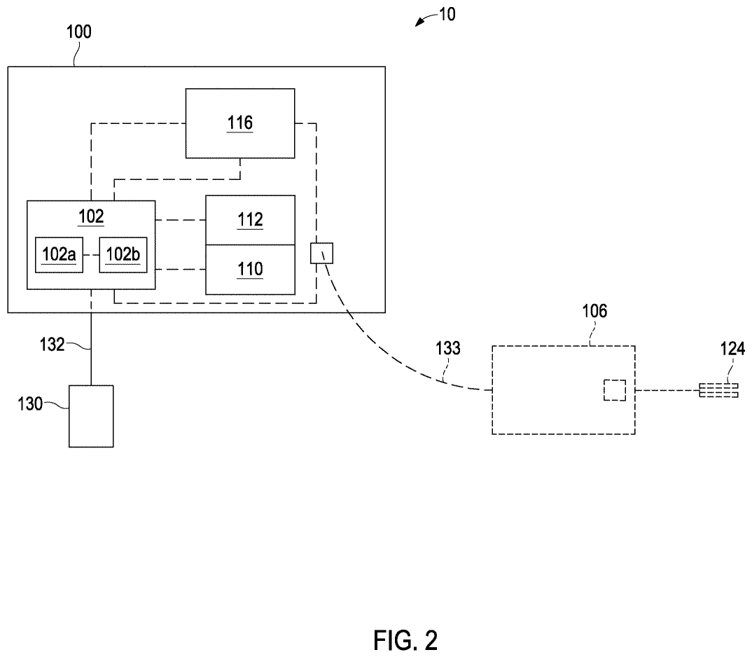

In some embodiments, the surgical instrument 106 may include a motor 150 that is operable to transition the end effector 124 between the open and closed configurations. The motor 150 may be actuatable based on a user providing an input to the surgical instrument 106, such as via the trigger 145, or to a controller 102 (FIG. 2), such as via an input device 110 (FIG. 2), described in more detail below. The handpiece sensor 144 (e.g., the encoder) may be used to sense the rotational position of a shaft of the motor 150, which may be indicative of a rotational position of the clamp arm 142a. In other embodiments, the motor 150 is omitted and the end effector 124 is transitioned between the open and closed positions by a closure system that includes gears, tubes, links, and rods, as examples, that is responsive to a user rotating the trigger 145.

While the foregoing surgical system 10 includes a handheld RF surgical instrument 106, the surgical system 10 may include, in the alternative or in addition therewith, a robotic RF surgical instrument that is similar to the RF surgical instrument.

FIG. 2 is a schematic block diagram of the surgical system 10 of FIG. 1, in accordance with at least one aspect of the present disclosure. The generator 100 may comprise several separate functional elements, such as modules and/or blocks. Different functional elements or modules may be configured for driving the surgical instrument 106. For instance, the generator 100 may include an electrosurgery/RF drive circuit or “RF generator” 116 to provide RF bipolar energy to the RF surgical instrument 106 via a cable 133.

The electrosurgery/RF drive circuit 116 may generate a drive signal or signals with output power sufficient to perform bipolar electrosurgery using RF energy. In bipolar electrosurgery applications, the drive signal may be provided, for example, to the electrodes 125 of the clamp arms 142a,b. Accordingly, the generator 100 may be configured for therapeutic purposes by applying electrical energy to the tissue sufficient for treating the tissue (e.g., coagulation, cauterization, tissue welding). The generator 100 may also be configured for subtherapeutic purposes by applying electrical energy to the tissue for monitoring parameters of the tissue during a procedure, such as impedance, for example.

The generator 100 may include a controller 102 that is in operable communication with the RF generator 116 and may be operable to control the same. The generator 100 may be activated to provide the drive signal to the RF surgical instrument 106 in any suitable manner. For example, the generator 100 may be in communication with a foot switch 130 via a foot switch cable 132. A clinician may energize the electrodes 125 of the clamp arms 142a,b by depressing the foot switch 130. In addition, or instead of the foot switch 130, the RF surgical instrument 106 may utilize the energy button 135 positioned on the handpiece 107 that, when activated, may cause the generator 100 to energize the electrodes 125 of the clamp arms 142a,b.

The generator 100 may further include an input device 110 located on a front panel of the generator 100 console. The input device 110 may comprise any suitable device that generates signals that can be used by the generator 100 (e.g., by one or more controllers 102 contained in the generator 100) to control the operation of the generator 100 (e.g., operation of the electrosurgery/RF drive circuit 116). The input device 110 may include one or more of buttons, switches, thumbwheels, keyboard, keypad, touch screen monitor, pointing device, remote connection to a general purpose or dedicated computer, or any combination thereof. The input device 110 may comprise a suitable user interface, such as one or more user interface screens displayed on a touch screen monitor. Accordingly, by way of the input device 110, the user can set or program various operating parameters of the generator 100, such as, for example, current (I), voltage (V), frequency (f), and/or period (T) of a drive signal or signals generated by the electrosurgery/RF drive circuit 116.

The generator 100 may also include one or more output devices 112, such as an output indicator, located, for example, on a front panel of the generator 100. The output device 112 can include one or more devices for providing a sensory feedback to a user. Such devices may comprise visual feedback devices or modules (e.g., incandescent lamps, LEDs, graphical user interface, display, analog indicator, digital indicator, bar graph display, digital alphanumeric display, liquid crystal display (LCD) screen, light emitting diode (LED) indicators), audio feedback devices or modules (e.g., a speaker, buzzer, audible, computer generated tone, computerized speech, voice user interface (VUI) to interact with computers through a voice/speech platform), or tactile feedback devices or modules (e.g., one or motors or any type of vibratory feedback, haptic actuator).

The controller 102 may include a processor 102a and a memory 102b comprising any storage media readable by the processor 102a. The memory 102b may store software or software instructions executable by the processor 102a. Based on instructions provided by the software, the controller 102 may be configured to control various aspects of the system 10, such as the output of the output device 112, a voltage, current, and/or frequency provided to the electrodes 125 via the electrosurgery/RF drive circuit 116, or a position of the knife of the RF surgical instrument 106, or combinations thereof.

### Concept 1: Tissue Compression Visualized in HUD

In an example operation, a user, such as a surgeon, may desire to seal and cut the tissue of a patient. The user may first position (locate) the tissue between the clamp arms 142a,b of the end effector 124. When satisfied with the position of the tissue, the user may squeeze (pull) the trigger 145 to transition the clamp arms 142a, b from the open configuration toward the closed configured to grasp the tissue therebetween.

According to “Histological Evaluation and Optimization of Surgical Vessel Sealing Systems to Lathrop et al (2017)”, which is hereby incorporated by reference in its entirety herein, “vapor formation may occur during the sealing process. In the thermally treated zone of the sealed vessel, if there is a large, interconnected field of vacuoles it is likely to provide an easy failure point if the vessel was pressurized. Large vacuoles may weaken the tunica adventitia, potentially enabling seal failure through the vessel wall.” The formation of these vacuoles and their subsequent bursting can result in rapid changes in the jaw gap during sealing. This can cause the strain rate adjacent the jaws in the adventitia to be very high, resulting in much higher stresses than if there was no vapor damage.

Blood vessels exhibit viscoelastic properties and are therefore strain-rate sensitive. Strain-rate effects mean the resulting stress on a body is dependent on the rate at which the deformation is applied. Low deformation (strain) rates correspond to lower stresses whereas higher deformation (strain) rates correspond to higher stresses. The adventitia, like all collagens, exhibits this behavior.

Prior to applying bipolar energy to the tissue, for optimal hemostasis performance, it is desirable to allow an amount of time to elapse, such as 5, 10, or 15 seconds, thereby allowing the tissue grasped between the jaws to relax and reach a viscoelastic “steady state”. In practice, typical clinical use includes no waiting time to allow for these viscoelastic effects to reach a “steady state”. Delivering therapeutic levels of energy to the tissue before it has reached its viscoelastic “steady state” may cause higher strain rates and therefore may cause higher stresses on the tissue within and immediately adjacent to clamp arms 142a,b of the end effector 124. However, the amount of time to reach the steady state is generally variable, where a first type of tissue may require a different amount of time (more or less time) than a second type of tissue different than the first type. It is generally desirable to complete the surgical procedure as quickly and efficiently as possible, such as to reduce the amount of time that the patient is under anesthesia. Therefore, rather than waiting a predetermined amount of time, systems and methods for providing a user with an indication of when clamped tissue has reached a steady state are desired.

As provided herein, determining the amount of time allowed to elapse prior to energizing the electrodes 125 can be accomplished by sensing the viscoelastic properties of the tissue over time, such as by using the sensor 141 (FIG. 1), to measure the change in gap or angle between the clamp arms 142a,b over time. This gap or angle, in combination with forces being applied to the tissue by the surgical instrument 106, as measured by the sensor 143, can be used to calculate the viscoelastic properties of the tissue, such as strain on the tissue, density, spring constant, thickness, and Young's Modulus.

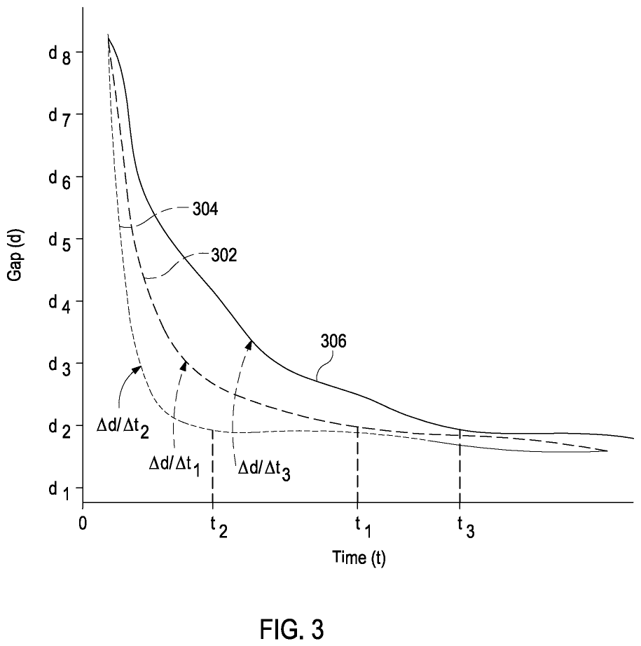

FIG. 3 provides an example graph 300 of gap distance between the clamp arms 142a,b (FIG. 1) over time. The gap distance may be measured by the first sensor 141 (FIG. 1) and elapsed time may be measured by a timer (not shown) of the controller 102 (FIG. 2).

With reference to FIGS. 1 and 3, the controller 102 may monitor, the rate of change of the gap distance between the clamp arms 142a,b, such as the rate of change of the gap distance while the clamp arms 142a,b are being actively approximated, toward the clamped position by the user pulling the trigger 145 and/or after the user has stopped squeezing the trigger 145, thereby allowing the tissue to relax and reach a steady state. The controller 102 may also detect an elapsed amount of time taken for the tissue between the clamp arms 142a,b to reach the steady state. The controller 102 may determine that the tissue has reached a steady state based on the rate of change of the gap distance reaching, or dropping below, a rate of change threshold, which may be stored in the memory 102b of the controller 102. Based on the rate of change of the gap distance and/or the amount of time taken to reach the steady state, the controller 102 may also determine a condition of the tissue grasped between clamp arms 142a,b.

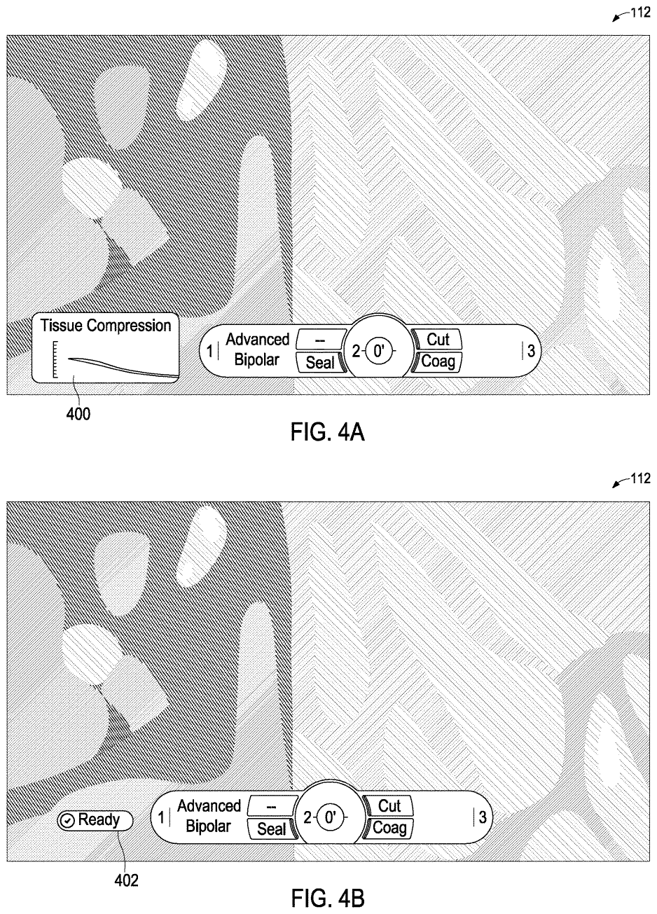

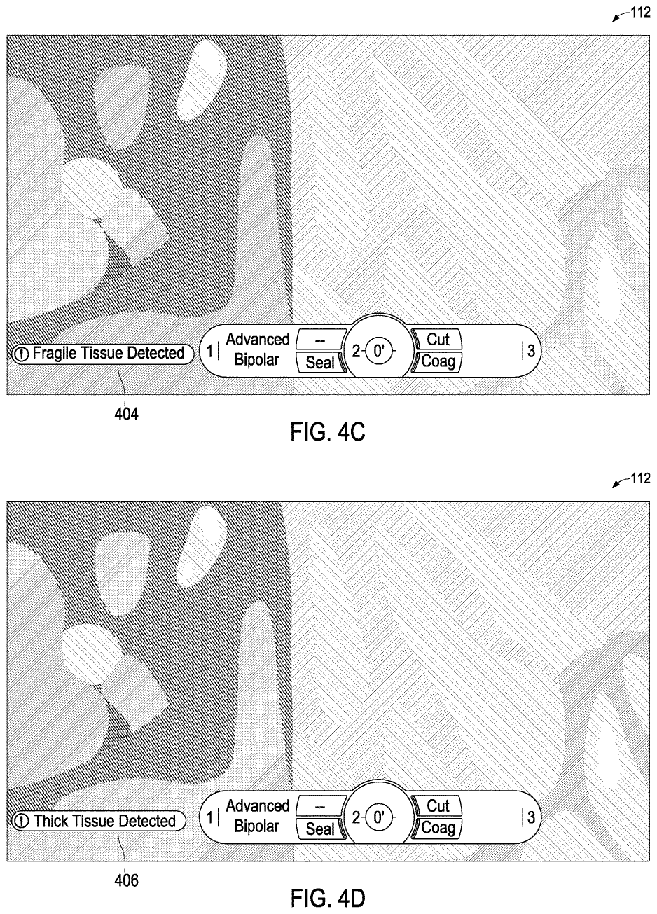

In a first example operation, a user may grasp a first type of tissue 302 between the clamp arms 142a,b, such as by squeezing the trigger 145 of the RF surgical instrument 106. The controller 102 may monitor, using the sensor 141 and the timer of the controller 102, the gap measurement magnitude, the rate of change of the gap distance Δd/Δt₁, as well as determine the amount of time taken to reach a steady state t₁. Referring to FIG. 4A, while the controller 102 monitors the rate of change of the gap distance, the controller 102 may display, on the output device 112, a visual depiction 400 of the graph 300 so that the user can visualize, in real time, the rate of change of the gap distance.

The memory 102b of the controller 102 may store therein a look-up table that correlates rates of change of gap distance and time taken to reach a steady state to conditions of tissue. As shown in FIG. 3, in the first example operation, the first type of tissue 302 may have a first or “normal” rate of change Δd/Δt₁ when the tissue 302 is grasped by the clamp arms 142a,b and may reach the steady state in a first amount of time t₁. The controller 102 may determine, based on the data in the lookup table, that the first rate of change Δd/Δt₁ and first amount of time t₁ correlates to tissue that is “normal” or suitable to be sealed and cut with the surgical instrument 106. Accordingly, with reference to FIG. 4B, based on the controller 102 determining that the tissue 302 has reached a steady state, the controller 102 may display, on the output device 112, a visual indicator 402 that informs the user that the RF surgical instrument 106 may be actuated to seal and cut tissue, as described elsewhere herein. In addition, or in the alternative thereto, the controller 102 may actuate an audio module 112 positioned in the RF surgical instrument 106 or the generator 100 to provide an audible indication that the RF surgical instrument 106 may be actuated to seal and cut the tissue. In addition, or in the alternative thereto, the controller 102 may actuate a tactile module 112, such as a motor, which may be positioned in the RF surgical instrument 106 to provide tactile feedback to the user that the RF surgical instrument 106 may be actuated to seal and cut the tissue.

In a second example operation, a user may grasp a second type of tissue 304 between the clamp arms 142a,b, such as by squeezing the trigger 145 of the RF surgical instrument 106. The controller 102 may monitor, using the sensor 141 and the timer of the controller 102, the gap measurement magnitude, the rate of change of the gap distance Δd/Δt₂, as well as determine the amount of time taken to reach a steady state t₂. Similar to above, referring to FIG. 4A, while the controller 102 monitors the rate of change of the gap distance, the controller 102 may display, on the output device 112, a visual depiction 400 of the graph 300 so that the user can visualize, in real time, the rate of change of the gap distance.

As referenced above, the memory 102b of the controller 102 may store therein a look-up table that correlates rates of change of gap distance and time taken to reach a steady state to conditions of tissue. As shown in FIG. 3, in the second example operation, the second type of tissue may have a second or “rapid” rate of change Δd/Δt₂ different (greater) than the first rate of change Δd/Δt₁ when the tissue 304 is grasped by the clamp arms 142a,b and may reach the steady state in a second amount of time t₂ different (less) than the first amount of time t₁. The controller 102 may determine, based on the data in the lookup table, that the second rate of change Δd/Δt₂ and second amount of time t₂ correlates to tissue that is fragile. Accordingly, with reference to FIG. 4C, based on the controller 102 determining that the tissue 304 has reached a steady state, the controller 102 may display on the output device 112 a visual indicator 404 informing the user of the condition of the tissue 304, such as a message saying the tissue is fragile. In addition, or in the alternative thereto, the controller 102 may actuate an audio module or a tactile module, as described above, that may inform the user of the condition of the tissue 304. With this information, the user may then decide if they wish to actuate the surgical instrument 106 or reposition the clamp arms 142a,b to a new position.

In a third example operation, a user may grasp a third type of tissue 306 between the clamp arms 142a,b, such as by squeezing the trigger 145 of the RF surgical instrument 106. The controller 102 may monitor, using the sensor 141 and the timer of the controller 102, the gap measurement magnitude, the rate of change of the gap distance Δd/Δt₃, as well as determine the amount of time taken to reach a steady state t₃. Similar to above, referring to FIG. 4A, while the controller 102 monitors the rate of change of the gap distance, the controller 102 may display, on the output device 112, a visual depiction 400 of the graph 300 so that the user can visualize, in real time, the rate of change of the gap distance. The visual depiction 400 of the graph 300 may be provided prior to, at the start of, and/or during activation of the electrodes 125.

As referenced above, the memory 102b of the controller 102 may store therein a look-up table that correlates rates of change of gap distance and time taken to reach a steady state to conditions of tissue. As shown in FIG. 3, in the third example operation, the third type of tissue may have a third or “slow” rate of change Δd/Δt₃ different (less) than the first rate of change Δd/Δt₁ when the tissue 306 is grasped by the clamp arms 142a,b and may reach the steady state in a third amount of time t₃ different (less) than the first amount of time t₁. The controller 102 may determine, based on the date in the lookup table, that the third rate of change Δd/Δt₃ and third amount of time t₃ correlates to tissue that is thick. Accordingly, with reference to FIG. 4D, based on the controller 102 determining that the tissue 306 has reached a steady state, the controller 102 may display on the output device 112 a visual indicator 406 that informs the user of the condition of the tissue 306, such as a message saying the tissue is thick. In addition, or in the alternative thereto, the controller 102 may actuate an audio module or a tactile module, as described above, that may inform the user of the condition of the tissue 306. With this information, the user may then decide if they wish to actuate the surgical instrument 106 or reposition the clamp arms 142a,b to a new position.

While the foregoing description was provided with respect to the handheld RF surgical instrument 106, the principles are equally applicable to a robotic RF surgical instrument that may be coupled to a robotic surgical system. Example robotic systems are described in U.S. Pat. No. 11,424,027, titled “METHOD FOR OPERATING SURGICAL INSTRUMENT SYSTEMS”, which issued on Aug. 23, 2022, U.S. Pat. No. 11,980,366, titled “SURGICAL INSTRUMENT”, which issued on May 14, 2024, and U.S. Pat. No. 12,062,442, titled “METHOD FOR OPERATING SURGICAL INSTRUMENT SYSTEMS”, which issued on Aug. 13, 2024, all of which are hereby incorporated by reference in their entireties herein.

### Concept 2: Tissue Compression Profile Gauge

In an example operation, a user, such as a surgeon, may desire to seal and cut the tissue of a patient. The user may position (locate) the tissue between the clamp arms 142a,b of the end effector 124. When satisfied with the position of the tissue, the user squeezes (pulls) the trigger 145 to transition the clamp arms 142a,b from the open configuration toward the closed configured to grasp the tissue therebetween.

Prior to applying bipolar energy to the tissue, it is desirable to allow an amount of time to elapse, such as 5, 10, or 15 seconds, thereby allowing the tissue grasped between the jaws to relax and reach a viscoelastic steady state, as discussed elsewhere herein. However, while a user may generally know the type of tissue they are going to seal and cut (e.g., lung, liver, stomach), the user may not know the condition of the tissue, such as if the tissue is fragile or thick. Accordingly, systems and methods for providing a user with an indication of the condition of the tissue clamped between the clamp arms 142a,b are desired.

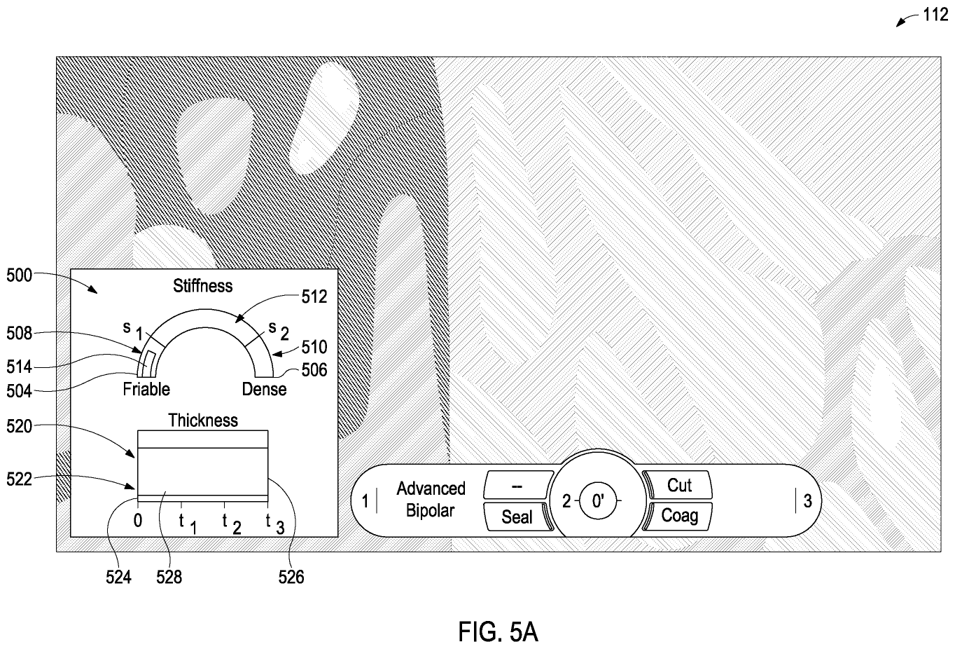

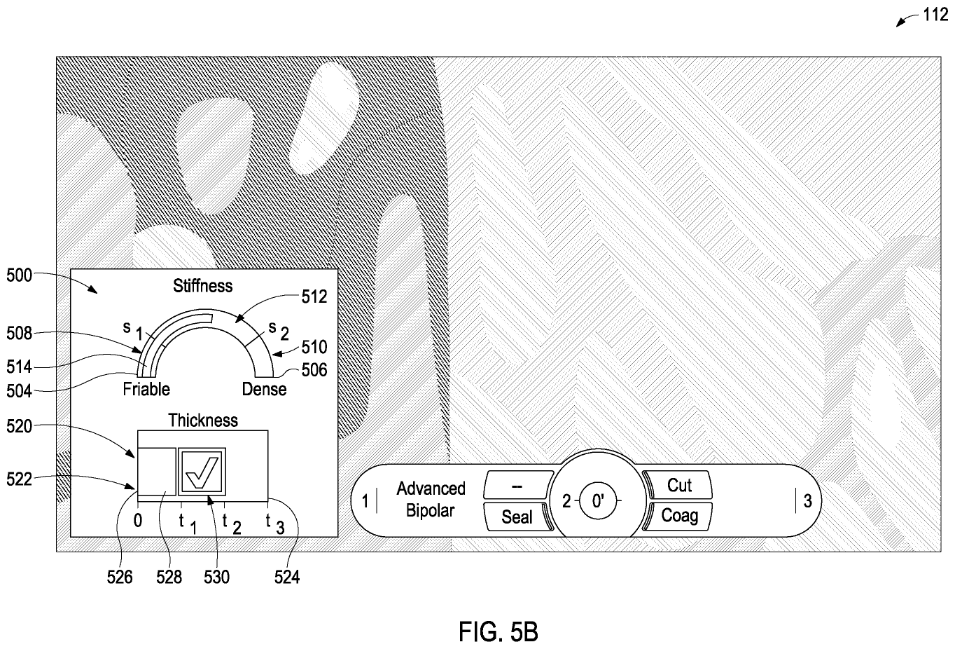

FIG. 5A is an example view of the output device 112 displaying first and second visual indicators 500, 520 that indicate a stiffness and a thickness, respectively, of tissue captured by the RF surgical instrument 106 (FIG. 1), in accordance with at least one aspect of the present disclosure.

As shown, the first visual indicator 500 may be a stiffness gauge 502 that includes a first or “minimum” end 504 and a second of “maximum” end 506. The stiffness gauge 502 may define a first of “friable” region 508 that extends from the first end 504 to a first stiffness value s₁ of the stiffness gauge 502 and a second or “dense” region 510 that extends from the second end 506 to a second stiffness value s₂ of the stiffness gauge 502 that is different (greater) than the first stiffness value s₁. The stiffness gauge 502 may further define a “normal” region 512 that extends between the first and second stiffness values s₁, s₂. The first visual indicator 500 may further include a dynamic indicator or “bar” 514 that moves along the stiffness gauge 502 between the first and second ends 504, 506 to provide an indication of a stiffness of tissue grasped by the clamp arms 142a,b (FIG. 1) of the RF surgical instrument 106 (FIG. 1), as will be discussed in more detail below. Notably, the bar 514 being located in the dense region 510 may indicate to a user that thick tissue is grasped by the clamp arm 142a,b, or that the clamp arms 142a,b have grasped an immovable, rigid, and/or extremely stiff object that is not compressible beyond a point (e.g., an underlying bougie, catheter, metal clip within the tissue).

The second visual indicator 520 may be a bar graph 522 that includes a first or “minimum” end 524 and a second or “maximum” end 526. The second visual indicator 520 may further include an indicator or “bar” 528 that dynamically moves along the bar graph 522 between the first and second ends 524, 526 to provide an indication of a thickness of tissue grasped by the clamp arms 142a,b (FIG. 1) of the RF surgical instrument 106 (FIG. 1), as will be discussed in more detail below.

With reference to FIGS. 1 and 5A, in an example operation, a user, such as a surgeon, may desire to seal and cut the tissue of a patient. The user may position (locate) the tissue between the clamp arms 142a,b of the end effector 124. When satisfied with the position of the tissue, the user may squeeze (pull) the trigger 145 to transition the clamp arms 142a,b from the open configuration toward the closed configured to grasp the tissue therebetween.

The controller 102 may detect a first gap distance between the clamp arms 142a,b when the clamp arms 142a,b makes initial contact with the tissue. For instance, the controller 102 may detect initial contact with the tissue based on the first sensor 141 or electrodes 125 contacting the tissue. The first gap distance may be indicative of an initial thickness of the tissue (i.e., thickness of tissue prior to the clamp arms 142a,b clamping the tissue therebetween). Based on the determined first gap distance, the controller 102 may adjust a location of the indicator 528 along the bar graph 522 to provide the user with a visual cue of the initial thickness of the tissue. For example, as shown in FIG. 5A, the clamp arms 142a,b may make initial contact with the tissue with a gap distance of t₃ defined therebetween, as measured by the sensor 141. Accordingly, the controller 102 may place the indicator 528 of bar graph 522 at a corresponding location there along, such as at the second end 528, as shown in FIG. 5A, thereby providing the user with a visual cue of the initial thickness of the tissue.

The user may continue to clamp the tissue with the clamp arms 142a,b, such as with the trigger 145. As the clamp arms 142a,b clamp the tissue, the controller 102 may dynamically adjust the location of the indicators 514, 528, thereby providing the user with a dynamic visual cue of the stiffness and the thickness of the tissue in real time.

The controller 102 may determine the stiffness of the tissue based on the gap distance between the clamp arms, as measured by the sensor 141, and based on the force applied to the tissue, as measured by the sensor 143. For instance, to determine the stiffness, the controller 102 may monitor the change in gap distance between the clamp arms 142a,b from the initial contact point with the tissue and with the amount of force applied to the tissue, such as using the force sensor 143. The controller 102 may utilize either a single metric (parameter), or a combination of measured metrics (parameters) to derive a stiffness value. These parameters may include stress on the tissue, strain on the tissue, change in density over time, spring constant, thickness, or Young's Modulus, or combinations thereof. Hooke's Law may be used to approximate the spring constant with the following expression:

$$\vert spring constant \vert = \vert \frac{F}{{t}_{intitial} - {t}_{final}} \vert$$

Where “F” is the force measured by the force sensor 143, tᵢₙᵢₜᵢₐₗ is the initial thickness of the tissue, as sensed by the sensor 141, and t_{final} is the dynamically changing thickness of the tissue, as sensed by the sensor 141. Alternatively, “F” may be a known constant force based on the closure mechanism of the device.

Stress, strain, and Young's Modulus (E) may be approximated by the following expression:

$$E = \frac{Stress}{Strain} = \frac{( \frac{F}{A} )}{( \frac{{t}_{intitial} - {t}_{final}}{{t}_{intitial}} )}$$

Where “F” is the force measured by the force sensor 143, A is the cross-sectional area of the tissue between the clamp arms 142a,b, tᵢₙᵢₜᵢₐₗ is the initial thickness of the tissue, as sensed by the sensor 141, and t_{final} is the dynamically changing thickness of the tissue, as sensed by the sensor 141. Alternatively, “F” may be a known constant force based on the closure mechanism of the RF surgical instrument 106. Change in density over time may be approximated by the following expression:

$$Δ ρ = {ρ}_{f} - {ρ}_{0} = \frac{m}{{V}_{f}} - \frac{m}{{V}_{0}} = \frac{m}{\text{.5} {b}^{2} w} ( \frac{1}{tan ( {θ}_{f} )} - \frac{1}{tan ( {θ}_{0} )} ) \approx ( \frac{1}{tan ( {θ}_{f} )} - \frac{1}{tan ( {θ}_{0} )} )$$

Where Δρ is change in density, mass m is assumed to be constant, b is the length of the jaws 142a,b, and w is the width of the jaws 142a,b. V₀ is the initial volume of tissue V_{f} is the final and t_{final} is the dynamically changing volume of the tissue, as calculated based on the angle sensed by the sensor 141. θ₀ is the initial angle (aperture) of the jaws 142a,b, θ_{f} is the dynamically changing angle of the jaws 142a,b, as measured by the sensor 141.

Based on the determined stiffness, the controller 102 may dynamically adjust the position of the stiffness indicator 514 while dynamically adjusting the position of the thickness indicator 528.

The user may continue to clamp the tissue with the clamp arms 142a,b, such as with the trigger 145. Once satisfied with the position of the clamp arms 142a,b, the user may allow the tissue to relax and reach a steady state, as described elsewhere herein. While allowing the tissue to reach the steady state, the controller 102 may continue to dynamically adjust the indicators 514, 528, such as in response to the changing or “relaxing” thickness of the tissue. As discussed elsewhere herein, the controller 102 may detect the tissue reaching the steady state by monitoring a rate of change of gap distance and detecting the rate of change reaching or dropping below a rate of change threshold.

Referring to FIG. 5B, based on the controller 102 detecting the tissue having reached the steady state, the controller 102 may further display, on the output device 112, a third visual indicator 530 that is indicative of the tissue reaching the steady state. The third visual indicator 530 may be, for example, a checkmark or other affirmative symbol located (positioned) on the bar graph 522 and that provides a visual cue to the user that the tissue has reached its steady state. Based on visualizing the third visual indicator 530, the user may inspect the location of the first indicator 514 to determine if the tissue is friable (indicator 514 located in the friable region 508), dense (indicator 514 located in the dense region 510), or normal (indicator 514 located in the normal region 512). Based on the knowledge provided by the visual indicators 500, 520, 530, the user can make an informed decision on whether to proceed with actuating the RF surgical instrument 106 or if the clamp arms 142a,b should be repositioned to a new location.

In some embodiments, the controller 102 may prevent the user from energizing the electrodes 125 of the clamp arms 142a,b based on the controller 102 determining that the tissue is friable or dense (indicator 514 located in friable or dense regions 508, 510). The aforementioned visual indicators may be provided prior to, at the start of, and/or during activation of the electrodes 125.

While the foregoing description was provided with respect to the handheld RF surgical instrument 106, the principles are equally applicable to a robotic RF surgical instrument that may be coupled to a robotic surgical system.

### Concept 2B: Tissue Compression Profile Gauge

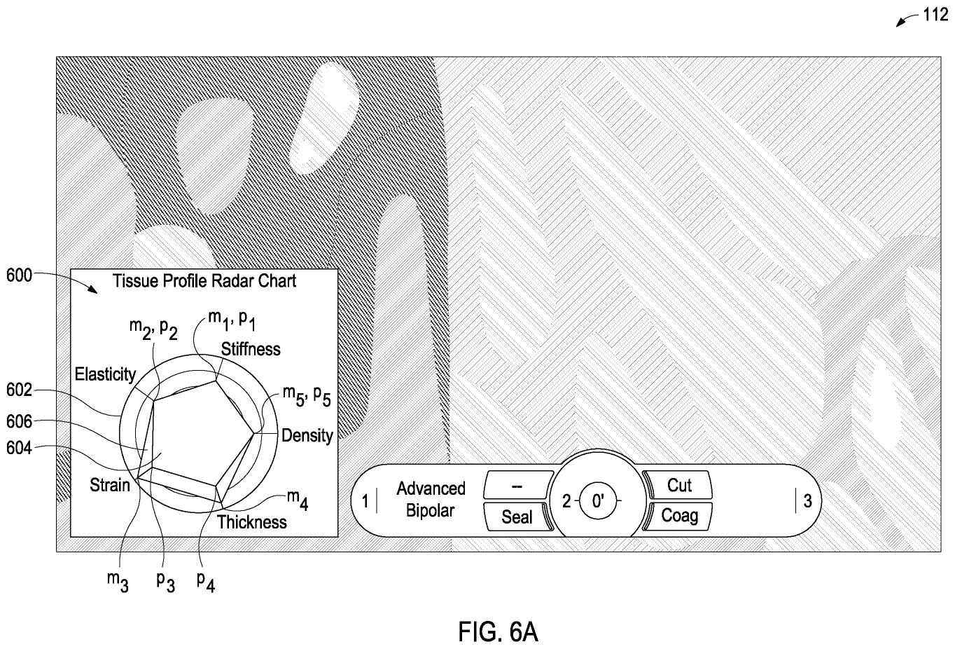

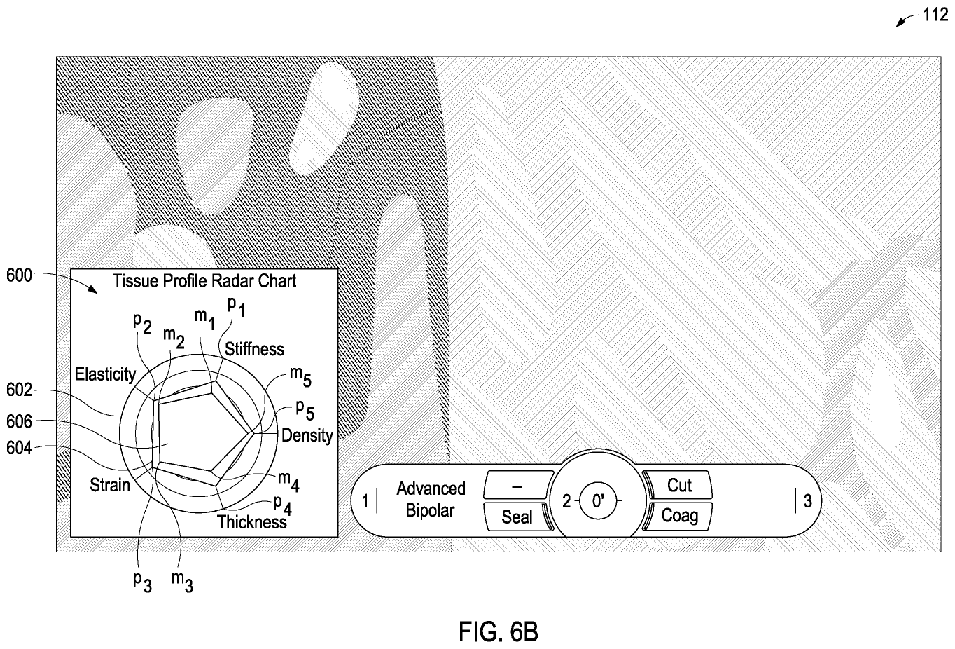

FIG. 6A is an example view of the output device 112 displaying a multiparameter visual indicator 600 that indicates various parameters of tissue captured by the RF surgical instrument 106 (FIG. 1), in accordance with at least one aspect of the present disclosure. The multiparameter visual indicator 600 may be displayed on the display in lieu of the first and second visual indicators 500, 520, or in combination therewith.

As shown, the visual indicator 600 may be a “radar-like” visual indicator that includes a circle 602 with a plurality of parameters, such as stiffness, elasticity, strain, thickness, and density, positioned about the circle 602 at spaced angular locations. The visual indicator 600 may include a target region 604 that is defined by threshold values of the parameters. The points of the target region 604 may be positioned at a location between the center of the circle 602 and a location at the perimeter of the circle 602 associated with the respective parameter. For instance, as shown in FIG. 6A, the target region 604 may be pentagonal in shape with the five points defined by a threshold stiffness p₁, a threshold elasticity p₂, a threshold strain p₃, a threshold thickness p₄, and a threshold density p₅, all of which may be stored in the memory 102b of the controller 102. The geometric shape of the target region 604 may be defined by the number of parameters. For instance, in the example of FIG. 6A, the target region is a pentagonal shape (five points) due to the five parameters of interest. Other target region shapes are possible, such as a square region (four parameters of interest), a triangular region (three parameters of interest), or an octagonal region (eight parameters of interest).

The visual indicator 600 may further include an indicator region 606 that is a corresponding shape to the target region 604 and that may dynamically change in size and shape based on the measured parameters, as will be discussed in more detail below. For instance, as shown in FIG. 6A, the indicator region 606 may, like the target region 604, be pentagonal in shape with the five points that are associated with a stiffness m₁, an elasticity m₂, a strain m₃, a thickness m₄, and a density m₅, all of which may be varied by the controller 102, such as via various sensors of the RF instrument 106, like the first and second sensors 141, 143.

With reference to FIGS. 1 and 6A, in an example operation, a user, such as a surgeon, may desire to seal and cut the tissue of a patient. The user may position (locate) the tissue between the clamp arms 142a,b of the end effector 124. When satisfied with the position of the tissue, the user may squeeze (pull) the trigger 145 to transition the clamp arms 142a,b from the open configuration toward the closed configured to grasp the tissue therebetween.

The controller 102 may detect the clamp arms 142a,b making initial contact with the tissue, such as based on the first sensor 141 or electrodes 125 contacting the tissue. Based on contacting the tissue, the controller 102 may measure and/or determine the various values of the parameters (e.g. stiffness, elasticity, strain, thickness, and density m₁-m₅) and overlay, on the target region 604, the indicator region 606 in a size corresponding to the values of the measured parameters m₁-m₅. The indicator region 606 may provide a visual cue to the user of the values of the various measured parameters m₁-m₅.

The indicator region 606 may be displayed (overlaid) in a first color, such as red, thereby indicating to the user that the tissue includes at least one parameter that fails to satisfy its corresponding threshold (e.g. the measured value is greater than the corresponding threshold value). For example, as shown in FIG. 6A, the tissue may be measured to have a strain and thickness m₃, m₄ that are greater than the strain threshold and thickness threshold p₃, p₄, respectively. Accordingly, the controller 102 may display the indicator region 606 as the first color.

The user may continue to clamp the tissue with the clamp arms 142a,b, such as with the trigger 145. As the clamp arms 142a,b clamp the tissue, the controller 102 may dynamically adjust the positions of the points m₁-m₅ according to the measured parameters, thereby changing the shape and size of the indicator region 606 relative to the target region 604, and thereby providing the user with a dynamic visual cue of the various parameters in real time.

Once satisfied with the position of the clamp arms 142a,b, the user may allow the tissue to relax and reach a steady state, as described elsewhere herein. While allowing the tissue to reach the steady state, the controller 102 may continue to dynamically adjust the positions of the points m₁-m₅, such as in response to the changing or “relaxing” tissue. As discussed elsewhere herein, the controller 102 may detect the tissue reaching the steady state by monitoring a rate of change of gap distance and detecting the rate of change reaching or dropping below a rate of change threshold.

Referring to FIG. 6B, based on the controller 102 detecting that all of the measured parameters m₁-m₅ are at, or below, their respective threshold values p₁-p₅, thereby causing the target region 604 to encompass the indicator region 606, the controller 102 may transition the indicator region 606 from the first color to a second color different than the first color, such as green, thereby providing a visual cue to the user than the tissue satisfies all of the thresholds of the parameters. Alternatively, based on the controller 102 detecting that at least one of the parameters m₁-m₅ is at, or above, its respective threshold value p₁-p₅, thereby causing the target region 604 to not completely encompass the indicator region 606, the controller 102 may maintain the indicator region 606 at the first color, thereby providing a visual cue to the user than the tissue fails to satisfy at least one of the thresholds of the parameters. Based on the knowledge provided by the visual indicator 600, the user can make an informed decision on whether to proceed with actuating the RF surgical instrument 106 or if the clamp arms 142a,b should be repositioned to a new location.

In some embodiments, the controller 102 may prevent the user from energizing the electrodes 125 of the clamp arms 142a,b based on at least one of the parameters of interest failing to reach, or fall below, its respective threshold.

While the foregoing description was provided with respect to the handheld RF surgical instrument 106, the principles are equally applicable to a robotic RF surgical instrument that may be coupled to a robotic surgical system.

### Concept 3: Advanced Sealing Mode for ABP

In an example operation, a user, such as a surgeon, may desire to seal and cut the tissue of a patient. The user may position (locate) the tissue between the clamp arms 142a,b of the end effector 124. When satisfied with the position of the tissue, the user may squeeze (pull) the trigger 145 to transition the clamp arms 142a,b from the open configuration toward the closed configured to grasp the tissue therebetween.

Prior to applying bipolar energy to the tissue, it is desirable to allow an amount of time to elapse, such as 5, 10, or 15 seconds, thereby allowing the tissue grasped between the clamp arms 142a,b to relax and reach a “steady state”, as discussed elsewhere herein.

However, the amount of time taken to reach the steady state may be variable between different types of tissue. For instance, a first type of tissue may require a first amount of time and a second type of tissue may require a second amount of time different than the first amount of time. Therefore, rather than waiting a predetermined amount of time, systems and methods for determining the optimal time to energize the RF surgical instrument 106 are desired.

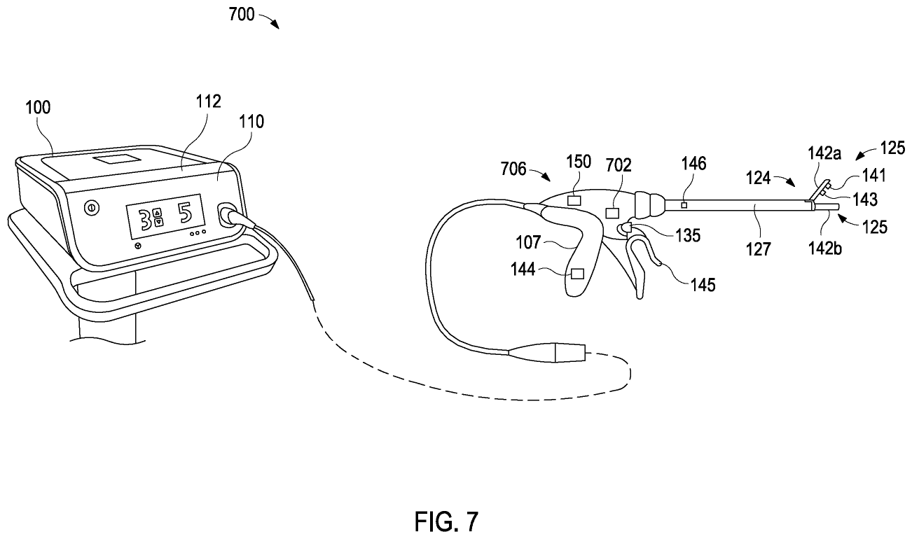

FIG. 7 illustrates a surgical system 700 comprising a generator 100 usable with an RF surgical instrument 706, in accordance with at least one aspect of the present disclosure. The surgical system 700 may may be similar in some respects to the surgical system 10 of FIG. 1, and therefore may be best understood with reference thereto, where like numerals will correspond to like components not described again in detail.

Unlike the RF surgical instrument 106, the RF surgical instrument 706 may include an advanced energy button 702 that may be in operable communication with the controller 102, such as via the wire 133 (FIG. 2), and may be actuated to initiate an advanced sealing algorithm, as will be discussed in more detail below. In the alternative, or in combination therewith, the system 700 may include a second footswitch (not shown) similar to the footswitch 130 (FIG. 2) and that may be actuated by the user to initiate the advanced sealing algorithm. In the alternative, or in combination therewith, the controller 102 may initiate the advanced sealing algorithm using a software-based mode selection that changes the functionality of one of the actuators, like the energy button 135 or the footswitch 130. For instance, the controller 102 may transition the RF surgical instrument 106 between a first mode and a second mode. In the first mode, interacting with the energy button 135 causes the RF surgical instrument 106 to apply energy to tissue, as described elsewhere herein. In the second mode, interacting with the energy button 135 causes the controller 102 to initiate the advanced sealing algorithm. The controller 102 may switch the RF surgical instrument 106 between the first mode and the second mode based on a user providing an input to the controller 102, such as via the input device 110.

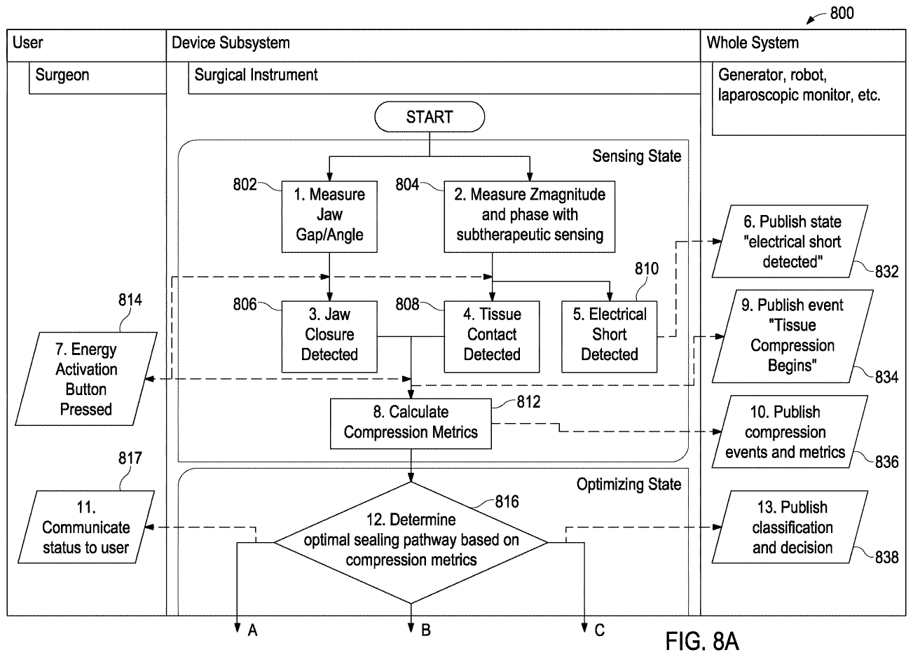

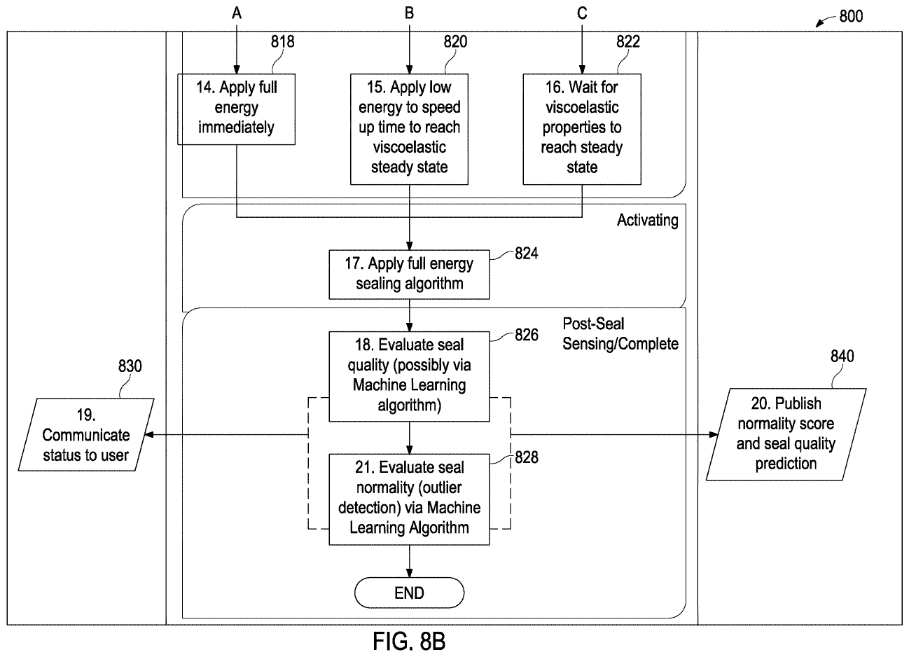

FIGS. 8A and 8B provide a schematic flow diagram 800 of an example method for controlling the surgical system 700 of FIG. 7, in accordance with at least one aspect of the present disclosure. The flow diagram 800 may be embodied as an algorithm (the aforementioned “advanced sealing algorithm”), stored in the memory 102b (FIG. 2) of the controller 102 (FIG. 2) and may be executed by the processor 102a (FIG. 2) of the controller 102, such as based on an input provided to the controller 102 by a user, such as at the input device 110 (FIG. 2).

With reference to FIGS. 7 and 8A, the method 800 may include a user positioning (locating) tissue between the clamp arms 142a,b of the end effector 124. The method 800 may further include measuring the jaw gap (or angle) between the clamp arms 142a,b, as at step 802. For instance, the controller 102 may measure the gap distance between the clamp arms 142a,b using the sensor 141.

The method 800 may further including measuring an impedance magnitude (Zmagnitude) and a phase with subtherapeutic sensing, as at step 804. For instance, as discussed elsewhere herein, the generator 100 may be configured for sub-therapeutic purposes, which may include by applying electrical energy to tissue from the electrodes 125 for monitoring parameters of the tissue, such as an impedance thereof. Accordingly, at step 804, the controller 102 may apply a subtherapeutic current from the electrodes 125 to measure the impedance magnitude (Zmagnitude) and phase.

The method 800 may further include detecting closure of the jaws, as at step 806. For instance, the controller 102 may detect closure of the clamp arms 142a,b based on detecting the user squeezing (pulling) the trigger 145, thereby causing the clamp arms 142a,b to transition from the open configuration towards the closed configuration.

The method 800 may further include detecting tissue contact, as at step 808. For instance, as discussed elsewhere herein, the controller 102 may detect the clamp arms 142a,b making contact with the tissue based on the first sensor 141 or electrodes 125 contacting the tissue. The gap distance sensed by the first sensor 141 at the initial tissue contact may be indicative of an initial thickness of the tissue (i.e., thickness of tissue prior to the clamp arms 142a,b clamping the tissue therebetween).

The method 800 may further include detecting an electrical short, as at step 810. For instance, the controller 102 may detect a short circuit between the electrodes 125, which may indicate that too thin or not enough tissue has been captured between the clamp arms 142a,b or that a staple or clip is present between the electrodes 125. Accordingly, the controller 102 may provide an indication to the user, such as via the output device 112, that the clamp arms 142a,b need to be repositioned.

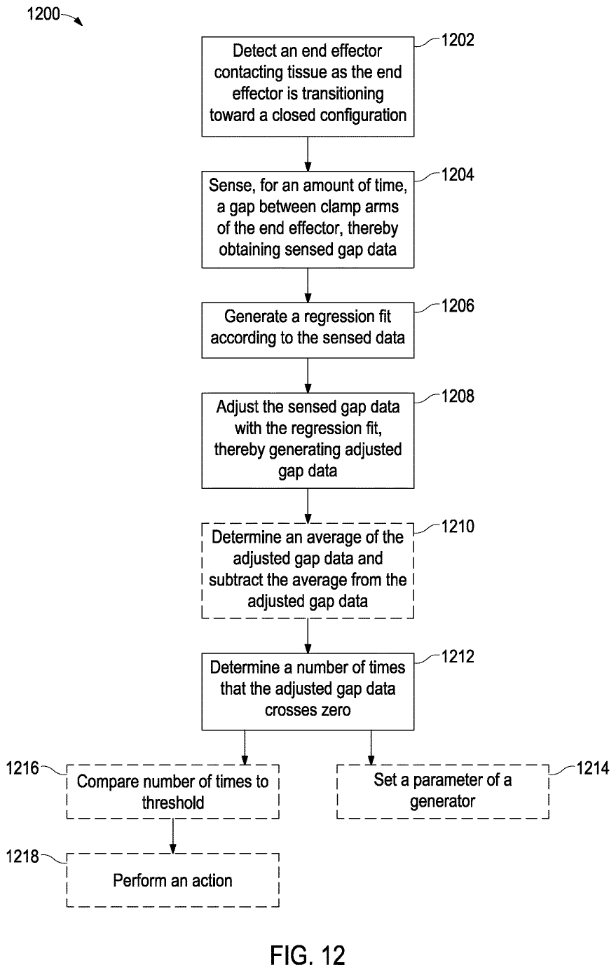

The method 800 may further include calculating compression metrics, as at step 812. For instance, as discussed elsewhere herein, the controller 102 may sense and/or determine various parameters about the tissue, such as the thickness, stiffness, elasticity, density, and/or strain of the tissue using the various sensors of the system 700, such as the first and second sensors 141, 143. In some embodiments, calculating compression metrics may include performing at least part, or all, of the flow diagram 1200 (FIG. 12), discussed elsewhere herein.

The method 800 may further include detecting activation of the advanced energy button 702, as at step 814. For instance, the controller 102 may detect the user actuating (pressing) the advanced energy button 702, such as prior to steps 806, 808, or 812. However, the controller 102 may detect the user pressing advanced energy button 702 at other times, such as before or after any of steps 802-812.

The method 800 may further include determining an optimal sealing pathway based on the compression metrics, as at step 816. For instance, based on the compression metrics determined at step 812, the controller 102 may selectively determine the next step of the method 800. The method 800 may further include communicating the selected step, as at step 817. For instance, the controller 102 may display, on the output device 112, which of the following step of the method 800 is to be performed. In addition, or in the alternative thereto, the controller 102 may actuate an audio module 112 or a tactile module 112 that informs the user which of the following step of the method 800 is to be performed.

For instance, based on first determined compression metrics, referring now to FIG. 8B, the method 800 may proceed to step 818, at which the controller 102 may immediately, and automatically (i.e., without user intervention), apply a first or “maximum” amount of energy to the tissue, such as prior to the tissue reaching a steady state. The controller 102 may proceed to step 818 in situations where the tissue is a smaller vessel that does not typically need to reach a steady state until energy can be applied safely thereto. The controller 102 may also proceed with step 824 and continue applying the first amount of energy to the tissue.

Based on the second determined compression metrics being different than the first determined compression metrics, the method 800 may proceed to step 820, at which the controller 102 may automatically (i.e., without user intervention) apply a second or “low” energy less than the first energy to the tissue as the tissue is relaxing and transitioning to the steady state. Applying the second energy to the tissue may aid in reducing the amount of time it takes for tissue, such as thick tissue, to reach the steady state and may lower the steady state jaw gap magnitude, such that the jaws 142a,b are closer together when the steady state is reached. The second energy may be pulsed, applied at a predetermined frequency, or applied continuously, or combinations thereof, during step 820. Based on the controller 102 detecting the tissue having reached the steady state, the controller 102 may proceed to step 824 and apply the first amount of energy to the tissue from the electrodes 125.

Based on third determined compression metrics different than the first and second determined compression metrics, the method 800 may proceed to step 822, at which the controller 102 may abstain from applying energy until the tissue reaches the steady state. For instance, as discussed elsewhere herein, the controller 102 may determine that the tissue has reached the steady state by monitoring the rate of change of the gap distance, via first sensor 141 and the timer of the controller 102, and detecting the rate of change of the gap distance reaching, or dropping below, a rate of change threshold, which may be stored in the memory 102b of the controller 102. Based on the controller 102 detecting the tissue reaching the steady state, the controller 102 may proceed to step 824 and apply the first amount of energy to the tissue from the electrodes 125.

The first, second, and third compression metrics, referenced herein above, may be stored in the memory 102b of the controller 102 and may be retrievable during the method 800. The compression metrics may be in the form of a lookup table and may be correlated to certain types of tissue.

The method 800 may further include evaluating the quality of the seal, as at step 826, and evaluating the seal normality, as at step 828. For instance, the controller 102 may apply a subtherapeutic amount of energy to the heated tissue to determine the impedance thereof, which may be correlated to the quality and normality of the seal. The quality and the normality of the seal may be evaluated using machine learning. The method 800 may further include communicating the quality and the normality of the seal, as at step 830. For instance, the controller 102 may display, on the output device 112, the quality and the normality of the seal for the user to view. In addition, or in the alternative thereto, the controller 102 may actuate an audio module 112 or a tactile module 112 that informs the user of the quality and the normality of the seal.

The method 800 may further include publishing various events of the method 800 to the memory 102b of the controller 102. For instance, the method 800 may include publishing if an electrical short is detected, as at step 832, publishing when tissue compression begins, as at step 834, publishing the compression metrics and events, as at step 836, publishing the classification of tissue and selected next step of the method 800, as at step 838, and publishing the quality and the normality of the seal, as at step 840. Any of the other steps of the method 800 may also be published, such detecting the clamp arms 142a,b being closed, subsequent to step 806, or that tissue contact has been detected, subsequent to step 808.

Based on applying the first amount of energy to the tissue from the electrodes 125, as at step 824, the controller 102 may automatically deploy the knife to cut the heated tissue. In the alternative, the controller 102 may display, on the output device 112, a visual indicator (not shown) that informs the user that they may proceed with cutting the heated tissue. In addition, or in the alternative thereto, the controller 102 may actuate an audio module or a tactile module that informs the user that they may proceed with cutting the heated tissue.

The system 700 and method 800 may enable the controller 102 to determine various compression metrics of tissue captured between the clamp arms 142a,b of the RF surgical instrument 706 and automatically select an appropriate next step of the method 800, which may be automatically and immediately applying energy at a maximum power level (e.g., step 818), automatically applying energy at a reduced power level to decrease the time taken for the tissue to reach a steady state (e.g., step 820), or automatically applying energy after the tissue has reached the steady state (e.g., step 822). Accordingly, compared to waiting a predetermined amount of time for all types of tissue (e.g., 5, 10, or 15 seconds), the foregoing system 700 and method 800 may selectively apply energy quicker (e.g., step 818) where it may not be necessary to wait the predetermined amount of time or slower (e.g. step 822) where it may be necessary to wait longer than the predetermined amount of time to improve seal quality.

While the foregoing description was provided with respect to the handheld RF surgical instrument 106, the principles are equally applicable to a robotic RF surgical instrument that may be coupled to a robotic surgical system.

Concept 4: Estimating Tissue Stiffness from Ringing

Variations in tissue properties may directly influence treatment outcomes by RF surgical instruments, such as RF surgical instrument 106 (FIG. 1), and may lead to unintended tissue damage. Surgical systems, such as surgical system 10 (FIG. 1), often include sensors, such as first and second sensors 141, 143 (FIG. 1), for monitoring these tissue properties, which may be used to set one or more parameters of the RF surgical instruments, such as a power level thereof or an activation time thereof. However, monitoring some tissue properties, such as tissue stiffness, remains a challenge.

Accordingly, systems and methods for measuring stiffness of tissue are desired.

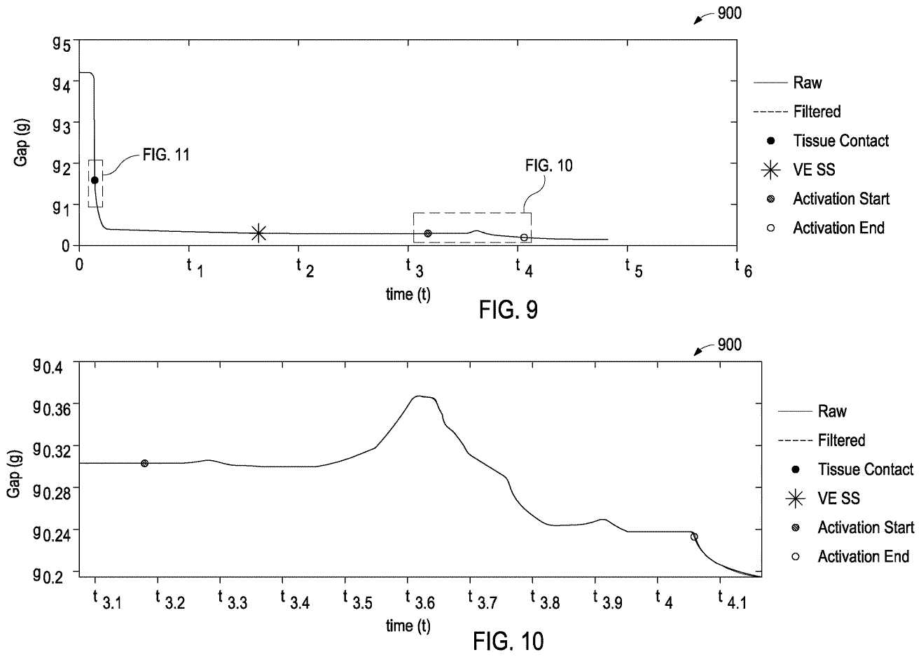

FIG. 9 is a graph of gap distance between the clamp arms 142a,b (FIG. 1) of the RF surgical instrument 106 (FIG. 1) over time during an example use of the RF surgical instrument 106. With reference to FIGS. 1 and 9, at time t₀, the clamp arms 142a,b are placed in the open configuration, thereby defining a gap therebetween of about g₄, as sensed by the first sensor 141. The clamp arms 142a,b are then transitioned (actuated) toward the closed configuration and eventually contact tissue positioned therebetween, which can be sensed by the second sensor 143. The clamp arms 142a,b continue to transition toward the closed configuration to clamp the tissue therebetween. The clamped tissue reaches a steady state between time t₁ and time t₂, with a gap distance of less than g₁ being defined between the clamp arms 142a,b. Energy is then supplied to the electrodes 125 of the clamp arms 142a,b shortly after time t₃ and energy is ceased to the electrodes 125 shortly after time t₄. The abbreviation “VE SS” in the legend is in reference to viscoelastic steady state.

FIG. 10 is an enlarged portion of the graph 900 of FIG. 9, as indicated by the dashed box in FIG. 9. In some instances, tissue may have a first or “large” stiffness such that, while energy is being supplied to the electrodes 125 of the clamp arms 142a,b, the gap between the first and second clamp arms 142a,b may increase, as illustrated at times t_{3.5} and t_{3.6}. Tissue with the first stiffness may not be properly sealable by the RF surgical instrument 106 using typical algorithms, or algorithms intended for sealing tissue with a second or “small” stiffness less than the first stiffness. Accordingly, it is desirable to determine stiffness of tissue to ensure that the tissue is properly sealed by the RF surgical instrument 106.

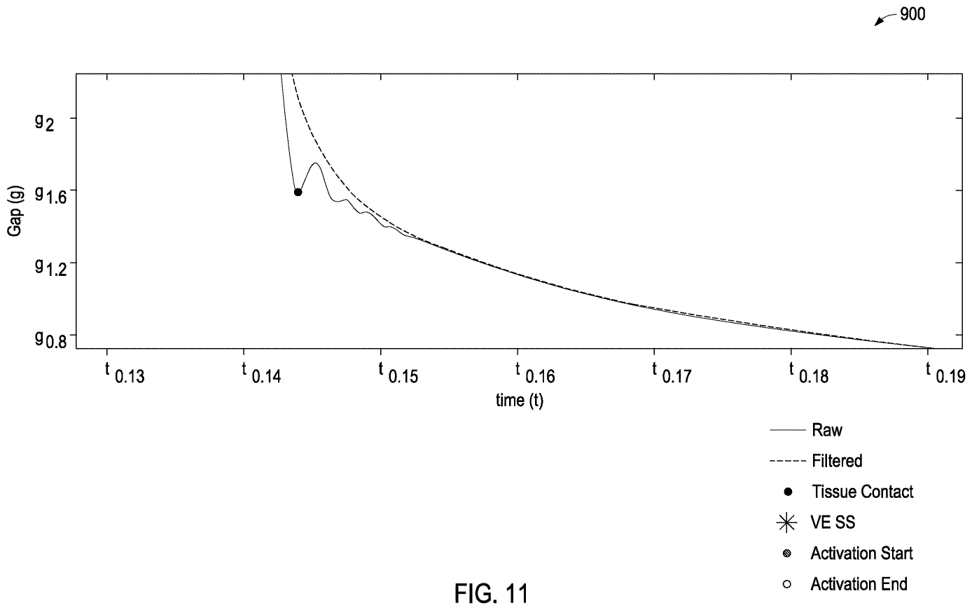

FIG. 11 is another enlarged portion of the graph 900 of FIG. 9, as indicated by the dashed box in FIG. 9. As illustrated, when the clamp arms 142a,b (FIG. 1) initially contact the tissue positioned therebetween (between times t_{0.14} to t_{0.15}), a “ringing” effect may be observed, in which the gap distance between the first and second jaws 142a,b oscillates. This observable ringing effect may be utilized to determine stiffness of the tissue, as explained in more detail below.

FIG. 12 is a schematic flowchart of an example method 1200 of determining stiffness of tissue, in accordance with at least one aspect of the present disclosure. The method 1200 may be embodied as an algorithm, stored in the memory 102b (FIG. 2) of the controller 102 (FIG. 2) and may be executed by the processor 102a (FIG. 2) of the controller 102, such as based on an input provided to the controller 102 by a user at the input device 110 (FIG. 2).

The method 1200 may include detecting an end effector contacting tissue as the end effector is transitioning toward a closed configuration, as at step 1202. For instance, with reference to FIGS. 1, 2, and 12, a user may decide to operate on a tissue using the RF surgical instrument 106. Accordingly, the user may grasp and manipulate the handpiece 107 to position the tissue between the clamp arms 142a,b of the end effector 124. Once satisfied with the position of the end effector 124, the user may actuate (squeeze) the trigger 145 to transition the end effector 124 from the open configuration toward the closed configuration. As the end effector 124 is transitioning toward the closed configuration, the controller 102 may detect the end effector 124 contacting the tissue utilizing the second sensor 143 and, more specifically, by sensing, via the second sensor 143, a spike (increase) in force, the spike being indicative of the second sensor 143 initially contacting the tissue. The controller 102 may also detect the end effector 124 contacting the tissue utilizing the electrodes 125 and, more specifically, by sensing, via a subtherapeutic signal applied by the electrodes 125, a change in measured impedance, the change being indicative of the electrodes 125 initially contacting the tissue.

In alternative embodiments, the motor 150 may be used to transition the end effector 124 toward the closed configuration, such as at a predefined force or torque, which may be stored in the memory 102b of the controller 102. As the motor 150 is transitioning the end effector 124 toward the closed configuration, the controller 102 may sense a velocity or an acceleration of the clamp arm 142a, such as with handpiece sensor 144, and may detect a change in velocity or acceleration, this change being indicative of the end effector 124 contacting tissue.

The method 1200 may further include sensing, for an amount of time, a gap between clamp arms of the end effector, thereby obtaining sensed gap data, as at step 1204. For instance, based on the controller 102 detecting the end effector 124 making contact with the tissue (step 1202), the controller 102 may sense, for an amount of time, a gap between the first and second clamp arms 142a,b, thereby obtaining (raw) sensed gap data. The amount of time may be a predefined amount of time and may be stored in the memory 102b of the controller 102. The amount of time may be about 50 ms, but could be less than 50 ms (e.g., 10 ms, 25 ms, 40 ms) or greater than 50 ms (e.g., 60 ms, 75 ms, 100 ms). The controller 102 may sense the gap with the first sensor 141. Accordingly, the system may utilize a first sensor (e.g., the sensor 141) during step 1202 and a second, different sensor (e.g., the sensor 143) during step 1204. Alternatively, the controller 102 may sense the gap with the same sensor used during step 1202 (e.g., the handpiece sensor 144). During the predefined amount of time, the controller 102 may sense, or observe, an oscillating (changing) gap, similar to what is shown in FIG. 11.

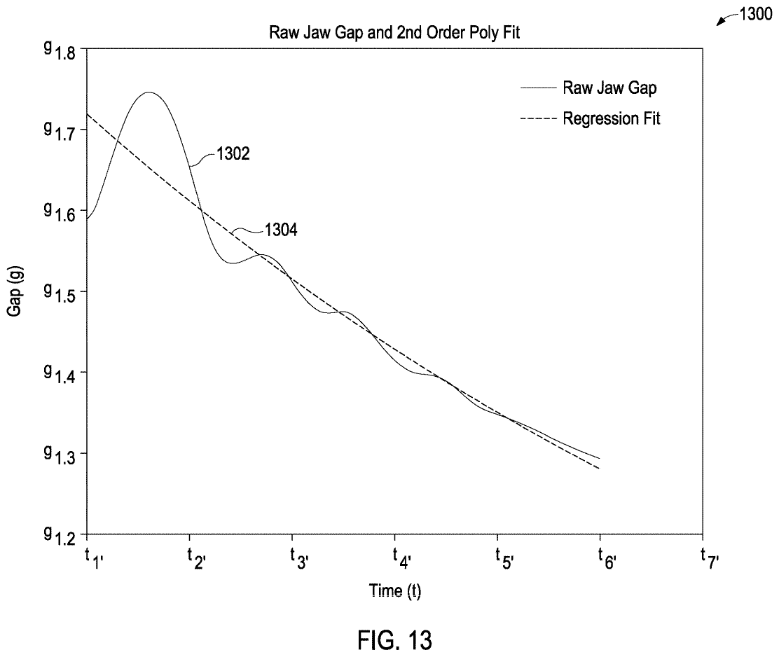

The method 1200 may further include generating a regression fit according to the sensed data, as at step 1206. For instance, with further reference to FIG. 13, the controller 102 may utilize the sensed gap data 1302 from the amount of time, as shown in graph 1300, and generate a regression fit 1304 to the sensed gap data 1302. The regression fit 1304 may be a polynomial and, more specifically, a second order polynomial (e.g., y=ax²+bx+c). Alternatively, the regression fit 1304 may be any mathematic equation, such as third order polynomial, exponential, logarithmic, piecewise, or the like.

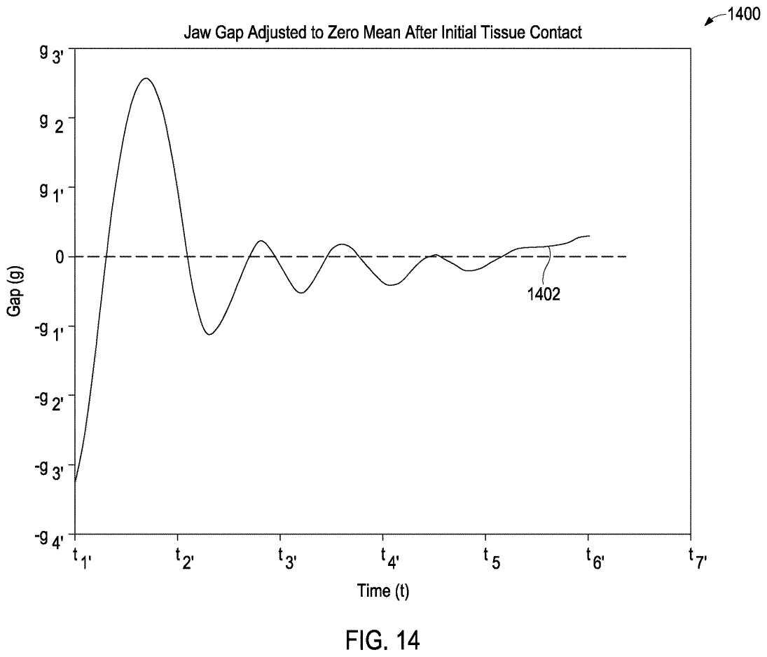

The method 1200 may further include adjusting the sensed gap data with the regression fit, thereby generating adjusted gap data, as at step 1208. For instance, the controller 102 may normalize the sensed gap data 1302 by subtracting the regression fit 1304, thereby generating adjusted gap data 1402, as shown in graph 1400 at FIG. 14.

The method 1200 may optionally further include determining an average of the adjusted gap data and subtracting the average from the adjusted gap data, as at step 1210. For instance, the controller 102 may further determine an average of the adjusted gap data and subtract this average from the adjusted gap data, thereby generating normalized gap data. Subtracting the average from the adjusted gap data may ensure that the normalized gap data has zero bias and with a mean value of 0.

The method 1200 may further include determining a number of times that the adjusted gap data (or normalized gap data if step 1210 is performed) crosses zero, as at step 1212. For instance, the controller 102 may detect (count) a number of times that the adjusted or normalized gap data crosses zero (e.g., transitions from a negative value to a positive value or a positive value to a negative value). As illustrated in the example provided in FIG. 14, the adjusted gap data 1402 crosses zero nine times.

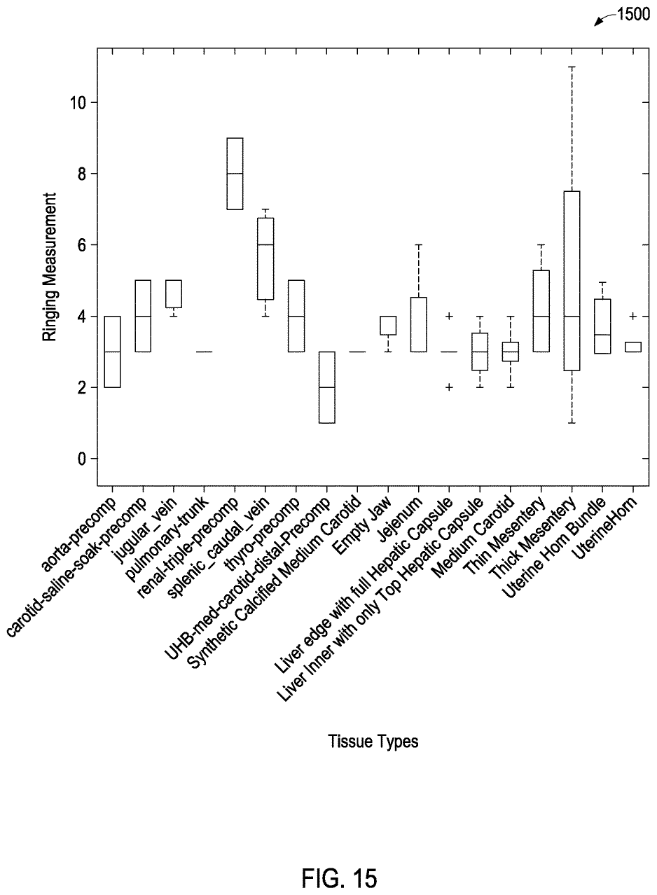

The number of times that the adjusted gap data crosses zero may be referred to as the “ringing estimate” or “ringing measurement”. The ringing estimate may be indicative of the stiffness of the tissue. For example, with reference to FIG. 15, a first or “large” ringing estimate may be indicative of tissue with a first or “high” stiffness (e.g., multiple renal arteries which may have a ringing estimate of about 8) and a second or “smaller” ringing estimate less than the first ringing estimate may be indicative of tissue with a second or “low” stiffness less than the first stiffness (e.g., thin mesentery which may have a ringing estimate of about 4).

In some embodiments, during step 1212, the controller 102 may determine a number of times that the adjusted or normalized gap data crosses zero at, or greater than, a rate of change threshold. For instance, the controller 102 may determine a rate of change of the adjusted gap data 1402 at each intersection with zero (e.g., g/t) and compare the rate of change to a known (predetermined) rate of change threshold, which may be stored in the memory 102b of the controller 102. The rate of change threshold may be 5°/sec, less than 5°/sec (e.g., 2°/sec, 3°/sec, 4°/sec) or greater than 5°/sec (e.g., 7°/sec, 8°/sec, 10°/sec). Comparing the rate of change to a rate of change threshold may prevent the controller 102 from recording lingering oscillations of the adjusted gap data 1402 around zero that may not accurately reflect the stiffness of the tissue.

In alternative embodiments, step 1208 may be omitted and step 1212 may include determining a number of times that the sensed gap data crosses the regression fit. For instance, the controller 102 may detect a number of times that the sensed gap data 1302 crosses the regression fit 1304 (e.g., when the difference between values of the regression fit 1304 from the corresponding values of the sensed gap data 1302 is zero). This number of times may be characterized as the ringing estimate.

The method 1200 may optionally include setting a parameter of a generator, as at step 1214. For instance, based on the ringing estimate, the controller 102 may set one or more parameters of the generator 100 coupled to the RF surgical instrument 106 or may select an energy delivery algorithm suitable for use with the tissue, or both. The parameters and algorithms may include current (I), voltage (V), frequency (f), or period (T), or combinations thereof, of a drive signal or signals that may be generated by the electrosurgery/RF drive circuit 116 of the generator 100.

The method 1200 may optionally include comparing the number of times to a threshold, as at step 1216. For instance, the controller 102 may compare the number of times (step 1212) to a threshold, which may be stored in the memory 102b of the controller 102. The method 1200 may optionally include performing an action based on the comparison of the number of times with the threshold, as at step 1218. For instance, if the number of times is at or greater than the threshold, the controller 102 may prevent the RF surgical instrument 106 from providing energy from the generator 100. The number of times being at or greater than the threshold may be indicative of tissue that is too stiff to be properly sealed by the RF surgical instrument 106. If the number of times is at or less than the threshold, the controller 102 may allow the RF surgical instrument 106 to provide energy from the generator 100. The number of times being at or less than the threshold may be indicative of tissue that is suitably stiff to be sealed by the RF surgical instrument 106. In the alternative, if the number of times is at or greater than the threshold, the controller 102 may allow the RF surgical instrument 106 to provide energy by the generator 100 and, if the number of times is at or less than the threshold, the controller 102 may prevent the RF surgical instrument 106 from providing energy by the generator 100.

The action may also include the controller 102 generating an alert, such as via the output device 112. The alert may be a visual alert that provides a visual cue to the user of the ringing estimate of the tissue. Based on the visual cue, the user may decide whether or not to seal the tissue with the RF surgical instrument 106.

In other embodiments, in lieu of steps 1206-1212, the method 1200 may include filtering the sensed gap data, such as with a low-pass filter, thereby generating filtered gap data. The method 1200 may then further include determining a number of times that the sensed gap data crosses the filtered gap data. This number of times may be characterized as the ringing estimate.

Accordingly, the foregoing method 1200 may enable a user and/or the controller 102 to determine a stiffness of tissue by observing oscillations in a gap between the clamp arms 142a,b over a period of time. The method 1200 may more accurately determine stiffness of tissue compared to traditional methods and may set one or more parameters of the generator 100 that is best suited for operating on said tissue, which may prevent unintended tissue damage, and/or achieve an intended therapeutic effect.

Concept 5: Seal Quality Prediction from Adventitia Fusing Energy

Referring again to FIG. 1, a typical vessel includes two layers-a tunica media or “media” layer and a tunica adventitia or “adventitia” layer surrounding the media layer. When energy is provided to the vessel with an RF surgical instrument, such as from the electrodes 125 of the clamp arms 142a,b of the RF surgical instrument 106, the media layer typically fractures and the opposing sides of the adventitia layer are fused together, thereby sealing a lumen of the vessel. The sealed vessel may then be cut with a knife incorporated in the RF surgical instrument.

In some situations, the vessel may not have been properly and/or completely sealed by the RF surgical instrument and, therefore, transecting the vessel could be potentially hazardous to the patient. Accordingly, systems and methods for increasing a user's knowledge about the status, or quality, of a seal prior to transection of a vessel are desired.

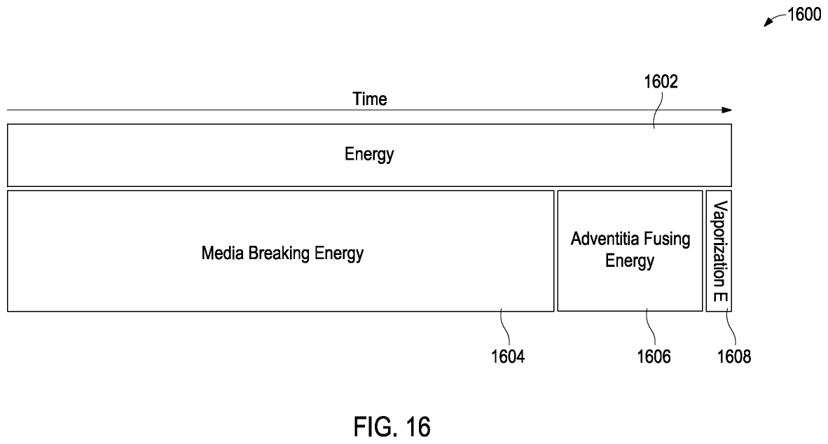

Referring now to FIG. 16, an example timeline 1600 is provided that illustrates energy applied to a vessel from the RF surgical instrument 106 (FIG. 1) over time. With reference to FIGS. 1 and 16, over the amount of time, a total amount of energy 1602 is provided to a vessel which may include first or “media breaking” energy 1604, second or “adventitia fusing” energy, and third or “vaporization” energy 1608. The media breaking energy 1604 may be the amount of energy provided by the RF surgical instrument 106 to break or “fracture” the media layer of the vessel. The adventitia fusing energy 1606 may be the amount of energy provided by the RF surgical instrument 106 to fuse the opposing sides of the adventitia layer together. The vaporization energy 1608 may be the amount of energy provided by the RF surgical instrument 106 after the vessel has reached the boiling point of water (i.e., 100° C. or 212° F.), thereby causing liquid within the vessel to vaporize. The inventors of the present disclosure have discovered that the amount of adventitia fusing energy 1606 provided to a vessel is indicative of the quality of the seal provided to the vessel from the RF surgical instrument 106. Accordingly, measuring the amount of adventitia fusing energy provided by the RF surgical instrument 106 may increase a user's knowledge about the status, or quality, of a seal prior to transection of the vessel.

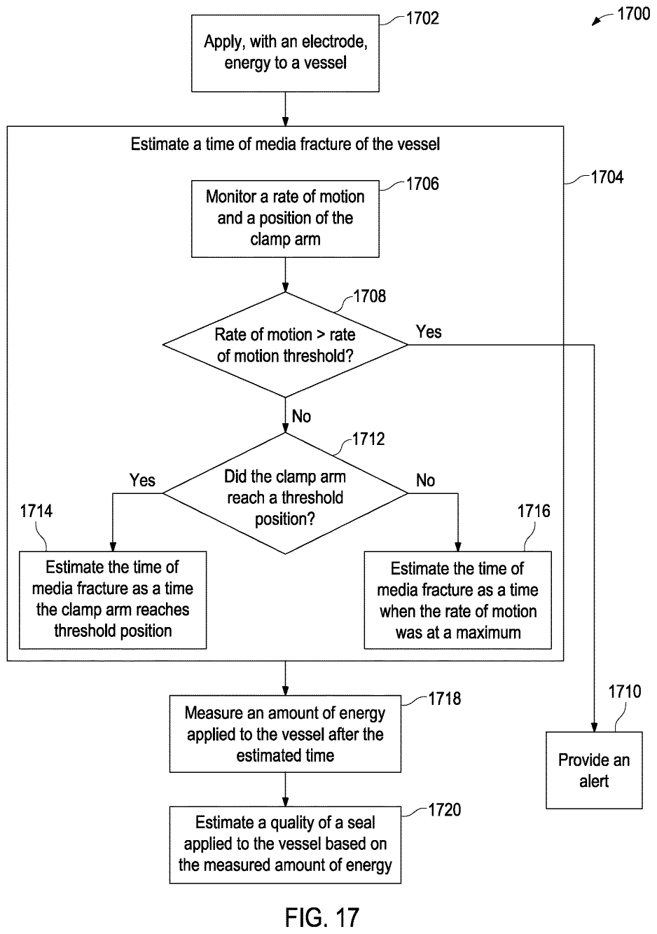

FIG. 17 provides a schematic flow diagram of an example method 1700 for controlling the surgical system 10 of FIG. 1, in accordance with at least one aspect of the present disclosure. The method 1700 may be embodied as an algorithm, stored in the memory 102b (FIG. 2) of the controller 102 (FIG. 2) and may be executed by the processor 102a (FIG. 2) of the controller 102, such as based on an input provided to the controller 102 by a user at the input device 110 (FIG. 2).

With reference to FIGS. 1, 2, and 17, the method 1700 may include applying, with an electrode, energy to a vessel, as at step 1702. For instance, a user of the RF surgical instrument 106 may determine that a vessel of a patient is to be sealed and transected. Accordingly, the user may maneuver the RF surgical instrument 106 to position the vessel between the clamp arms 142a,b. The user may then actuate the trigger 145 to cause the first clamp arm 142a to move (rotate) toward the second clamp arm 142b (manually or via the motor 150) to grasp the vessel therebetween. Based on the controller 102 detecting the end effector 124 contacting the vessel, such as via a change in impedance sensed by the first sensor 141 or electrodes 125, or a change in force sensed by the second sensor 143, the controller 102 may apply energy to the electrodes 125 from the RF generator 116.

The method 1700 may further include estimating a time of media fracture of the vessel, as at step 1704. For instance, the controller may estimate (determine) a point in time in which the media layer of the vessel is expected to have fractured. By estimating the time at which the media layer fractured, the controller 102 may conclude that the energy applied by electrodes 125 prior to that time was media breaking energy 1606 (FIG. 16), and after that time is adventitia fusing energy 1608 (FIG. 16) or vaporization energy 1610 (FIG. 16).

Estimating a time of media fracture of the vessel may include a plurality of sub-steps. The method 1700 may include monitoring a rate of motion and a position of the clamp arm, as at step 1706. For instance, the controller 102 may, using the first sensor 141 (e.g., the Hall-Effect sensor), sense the position of the first clamp arm 142a relative to the second clamp arm 142b as the first clamp arm 142a moves (rotates) toward the closed configuration to sense (determine) a gap therebetween. The controller 102 may use this positional information to calculate (determine) the rate of motion of the first clamp arm 142a.

The method 1700 may further include determining if the rate of motion of the first clamp arm 142a is greater than a corresponding rate of motion threshold, as at step 1708. For instance, the controller 102 may compare the sensed (monitored) rate of motion of the first clamp arm 142a to a rate of motion threshold, which may be stored in the memory 102b of the controller 102. Based on the sensed rate of motion reaching or exceeding the rate of motion threshold, the controller 102 may conclude that there was an uncontrolled rate of vessel collapse (e.g., the media layer of the vessel fractured too quickly) and, as such, the RF surgical instrument 106 may not be able to apply a proper seal to the vessel at the current location. Accordingly, the method 1700 may include providing an alert, as at step 1710. For instance, based on the controller 102 detecting the sensed rate of motion reaching or exceeding the rate of motion threshold, the controller 102 may provide an alert, such as an audible alert via the output device 112, a tactile alert via a tactile module (e.g., motor) in the handle 107, or a visual alert via the output device 112, or combinations thereof, thereby providing a cue to the user of the RF surgical instrument 106 that the end effector 124 needs to be repositioned to a new location on the vessel.

Based on the sensed rate of motion staying below the rate of motion threshold, the method 1700 may proceed to determine if the clamp arm reached a threshold position, as at step 1712. For instance, the controller 102 may compare the sensed (monitored) position of the first clamp arm 142a to a threshold position, which may be stored in the memory 102b of the controller 102. The threshold position of the first clamp arm 142a may correspond to a predefined gap between the first and second clamp arms 142a,b, which may be sensed by the first sensor 141 (e.g., the Hall-Effect sensor). The first clamp arm 142a may move (rotate) through a range of motion from the open configuration toward the closed configuration, and the threshold position of the first clamp arm 142a may be about 95% of the range of motion from the open configuration, or about 5% of the range of motion from the closed configuration. However, the threshold position may be greater than 95% of the range of motion from the open configuration (e.g., about 96%, 97%, 98% or 99%) or less than 95% of the range of motion from the open configuration (e.g., about 75%, 80%, 85%, or 90%).

Based on the clamp arm reaching the threshold position, the method 1700 may estimate the time of media fracture as the time the clamp arm reached threshold position, as at step 1714. For instance, based on the controller 102 detecting the first clamp arm 142a reaching the threshold position, the controller 102 may estimate that the media of the vessel fractured at, or around, the time that the first clamp arm 142a reached the threshold position. Based on the clamp arm failing to reach the threshold position, the method 1700 may proceed to estimate the time of media fracture as a time when the rate of motion was at a maximum, as at step 1716. For instance, the vessel located between the clamp arm 142a,b may be a “larger” vessel, thereby preventing, or hindering, the first clamp arm 142a from reaching the threshold position. Accordingly, the controller 102 may detect a point in time at which the rate of motion of the first clamp arm 142a was at a maximum value while applying energy to the vessel, and the controller 102 may estimate the time of media fracture at said time when the rate of motion was at a maximum. The controller 102 may conclude that the first clamp arm 142a may not reach the threshold position and proceed with estimating the time at step 1716 after a threshold amount of time has elapsed from the electrodes 125 beginning to apply energy (step 1702). The threshold amount of time may be stored in the memory 102b of the controller 102.

Alternatively, based on the clamp arm failing to reach the threshold position, the method 1700 may proceed to estimate the time of media fracture as a time when the rate of motion was at or below the rate of motion threshold for an elapsed predefined length of time. For instance, the vessel located between the clamp arm 142a, b may be a “larger” vessel, thereby preventing, or hindering, the first clamp arm 142a from reaching the threshold position. As such, the first clamp arm 142a may reach a “steady state”, which may be a state in which the rate of motion of the first clamp arm 142a remains at or below the rate of motion threshold. The controller 102 may detect that the first clamp arm 142a remained in the steady state for a threshold amount of time, which may be stored in the memory 102b of the controller 102. The controller 102 may estimate the time of media fracture as a time after the predetermined amount of time elapsing.

The method 1700 may further include measuring an amount of energy applied to the vessel after the estimated time, as at step 1718. For instance, based on the controller obtaining an estimated time of media fracture of the vessel (e.g., steps 1714 or 1716), the controller 102 may estimate the amount of energy applied to the vessel after the estimated time. The amount of energy applied to the vessel after the estimated time may be measured (monitored) by the controller 102 by measuring the amount of energy output by the RF generator 116, such as via a current, voltage, or power sensor in electrical communication with the generator 100.

As described elsewhere herein, energy applied to the vessel after the media breaking energy 1604 (FIG. 16) includes adventitia fusing energy 1606 (FIG. 16) and vaporization energy 1608 (FIG. 16). The controller 102 may determine a time at which the RF surgical instrument 106 transitions from applying adventitia fusing energy 1606 to vaporization energy 1608 by sensing a temperature of the vessel, such as with the temperature sensor 147, and comparing the sensed temperature to a temperature threshold, which may be stored in the memory 102b of the controller 102. The threshold temperature may be at, or about, the boiling temperature of water (i.e., 100° C. or 212° F.). The energy applied after the estimated time and with the sensed temperature being below the temperature threshold may correspond to adventitia fusing energy 1606 and energy applied after the estimated time and with the sensed temperature being at, or above, the temperature threshold may correspond to vaporization energy 1608.

The controller 102 may also determine a time at which the RF surgical instrument 106 transitions from applying adventitia fusing energy 1606 to vaporization energy 1608 by sensing the phase of the electrical signal provided to the electrodes 125, and comparing the sensed phase to a phase threshold, which may be stored in the memory 102b of the controller 102. The energy applied after the estimated time and with the sensed phase being below the phase threshold may correspond to adventitia fusing energy 1606 and energy applied after the estimated time and with the sensed phase being at, or above, the phase threshold may correspond to vaporization energy 1608.

The controller 102 may also determine a time at which the RF surgical instrument 106 transitions from applying adventitia fusing energy 1606 to vaporization energy 1608 by sensing the impedance of the vessel and comparing the sensed impedance to an impedance threshold, which may be stored in the memory 102b of the controller 102. The energy applied after the estimated time and with the impedance phase being below the impedance threshold may correspond to adventitia fusing energy 1606 and energy applied after the estimated time and with the sensed impedance being at, or above, the impedance threshold may correspond to vaporization energy 1608.

Based on the controller 102 obtaining an estimated time of media fracture of the vessel (e.g., steps 1714 or 1716), the controller 102 may also control the energy output by the RF generator 116, such as via a current, voltage, or power. The controller 102 may control the energy output by the RF generator 116 based on a position of the clamp arm 142a, such as initial position thereof (e.g., the open position) or based on a position of the clamp arm 142a when the clamp arm 142 contacts the vessel. The controller 102 may also control the energy output by the RF generator 116 based on the rate of motion of the clamp arm 142a. The energy output may be a ratio or multiplier determined (calculated) from the maximum rate of motion of the first clamp arm 142a and/or the initial position of the first clamp arm 142a.

The method 1700 may further include estimating a quality of a seal applied to the vessel based on the measured amount of energy, as at step 1720. For instance, based on the amount of energy measured (applied) to the vessel after the estimated time of media fracture (step 1718), the controller 102 may determine a quality, or completeness, or a seal applied to the vessel. The controller 102 may make its determination of the quality, or completeness, of the seal based on both the adventitia and vaporization energies 1606, 1608 (FIG. 16), based on the adventitia energy 1606 (FIG. 16) alone, or based on the vaporization energy 1608 (FIG. 16) alone.

In other embodiments, the method 1700 may further include measuring an amount of energy applied to the vessel prior to the estimated amount of time. For instance, based on the controller obtaining an estimated time of media fracture of the vessel (e.g., steps 1714 or 1716), the controller 102 may estimate the amount of energy applied to the vessel prior to the estimated time, where this estimated amount of energy corresponds to the amount of media breaking energy applied to the vessel. In such embodiments, the controller 102 may make its determination of the quality, or completeness, of the seal further based on the media breaking energy. For instance, the controller 102 may make its determination of the quality, or completeness, of the seal based on a magnitude and/or proportions of the media breaking, adventitia fusing, and vaporization energies applied to the vessel.

The controller 102 may compare the measured energy to an energy threshold range, which may be stored in the memory 102b, and which may include a first or “minimum” threshold, a second or “intermediate” threshold, and a third or “maximum” threshold. Based on the measured energy being at, or near, the minimum threshold, the controller 102 may conclude that the quality of the seal applied to the vessel was a “poor” quality seal. As such, the controller 102 may provide an alert (e.g., audible, visual, haptic) to the user indicating that the vessel should not be transected at the current location and that, instead, the end effector 124 should be repositioned at a new location on the vessel and the method 1700 should be repeated. In addition, the controller 102 may also prevent the user from actuating the knife to cut the vessel based on the determination of the seal being a poor quality seal.

Based on the measured energy being at, or near, the intermediate threshold, the controller 102 may conclude that the quality of the seal applied to the vessel was an “adequate” quality seal. As such, the controller 102 may provide an alert (e.g., audible, visual, haptic) to the user indicating that the vessel received enough energy to seal the vessel, but that the user should proceed with caution when transecting the vessel. In addition, the controller 102 may allow the user to actuate the knife to cut the vessel based on the determination of the seal being an adequate quality seal but may change (reduce) one of the operational parameters of the RF surgical instrument 106. For instance, based on the determination of the seal being an adequate quality seal, the controller 102 may reduce the speed at which the knife may travel, or may reduce the amount of force the knife can apply to the vessel before stalling.

Based on the measured energy being at, or near, the maximum threshold, the controller 102 may conclude that the quality of the seal applied to the vessel was a “good” quality seal. As such, the controller 102 may provide an alert (e.g., audible, visual, haptic) to the user indicating that the vessel received enough energy to seal the vessel and that the vessel can be transected. In addition, the controller 102 may allow the user to actuate the knife to cut the vessel based on the determination of the seal being a good quality seal at “normal” (not reduced) operational parameters of the RF surgical instrument 106, such as at a predefined speed or force, that may be stored in the memory 102b of the controller 102.

Accordingly, the foregoing method 1700 may increase a user's knowledge about the status, or quality, of a seal prior to transection of a vessel.

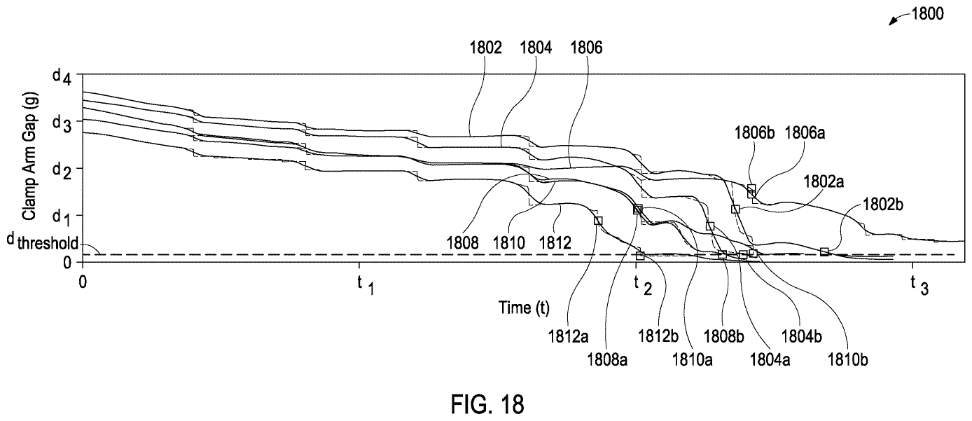

FIG. 18 is a graph 1800 illustrating a gap between the clamp arms 142a,b (FIG. 1) over time during example uses 1802, 1804, 1806, 1808, 1810, 1812 of the RF surgical instrument 106 (FIG. 1) sealing a vessel. The controller 102 may implement the method 1700 (FIG. 17) in connection with each of these examples uses 1802-1812 to obtain a status, or quality, of the seal for each example use 1802-1812.

For example, with reference to FIGS. 1, 2, 17, and 18, during the first example use 1802, the controller 102 may identify, using the first sensor 141 (e.g., the Hall-Effect sensor) that, at time point 1802a, the first clamp arm 142a rotated (moved) toward the closed configuration at a rate of motion that was greater than the rate of motion threshold (step 1708) and reached the threshold position d_{threshold} at time point 1802b (step 1712). Due to the controller 102 identifying a rate of motion of the first clamp arm 142a greater than the rate of motion threshold, the controller 102 may conclude that there was an uncontrolled rate of vessel collapse (e.g., the media layer of the vessel fractured too quickly) and, as such, the RF surgical instrument 106 may not have applied a proper seal to the vessel at the current location. As such, the controller 102 may provide an alert (step 1710) indicating the same to the user.

Similarly, during the second example use 1804, the controller 102 may identify, using the first sensor 141 (e.g., the Hall-Effect sensor) that, at time point 1804a, the first clamp arm 142a rotated (moved) toward the closed configuration at a rate of motion that was greater than the rate of motion threshold (step 1708) and reached the threshold position d_{threshold} at time point 1804b (step 1712). Due to the controller 102 identifying a rate of motion of the first clamp arm 142a greater than the rate of motion threshold, the controller 102 may conclude that there was an uncontrolled rate of vessel collapse (e.g., the media layer of the vessel fractured too quickly) and, as such, the RF surgical instrument 106 may not have applied a proper seal to the vessel at the current location. As such, the controller 102 may provide an alert (step 1710) indicating the same to the user.

During the third example use 1806, the controller 102 may identify, using the first sensor 141 (e.g., the Hall-Effect sensor) that the first clamp arm 142a did not rotate (move) toward the closed configuration at a rate of motion that was greater than the rate of motion threshold (step 1708), but rather, reached a maximum rate of motion at time point 1806a. Furthermore, the controller 102 may have identified that the first clamp arm 142a failed to reach the position threshold d_{threshold}. Accordingly, the controller 102 may estimate that the media of the vessel fractured at time point 1806b, which is the same or similar time point to time point 1806a (step 1716). Therefore, the controller 102 may measure the amount of energy applied to the vessel from time point 1806b, as described above, to determine a status, or quality, of the seal to the vessel.

During the fourth, fifth, and sixth example uses 1808, 1810, and 1812, the controller 102 may identify, using the first sensor 141 (e.g., the Hall-Effect sensor) that the first clamp arm 142a did not rotate (move) toward the closed configuration at a rate of motion that was greater than the rate of motion threshold (step 1708), but rather, reached a maximum rate of motion at time points 1808a, 1810a, and 1812a, respectively. However, the controller 102 may identify that the first clamp arm 142a reached the position threshold d_{threshold} at time points 1808b, 1810b, and 1812b, respectively. Accordingly, the controller 102 may estimate that the media of the vessel fractured at time points 1808b, 1810b, and 1812b for the fourth, fifth, and sixth example uses, respectively (step 1712). Therefore, the controller 102 may measure the amount of energy applied to the vessel from time points 1808b, 1810b, 1812b, as described above, to determine a status, or quality, of the seal to the vessel.

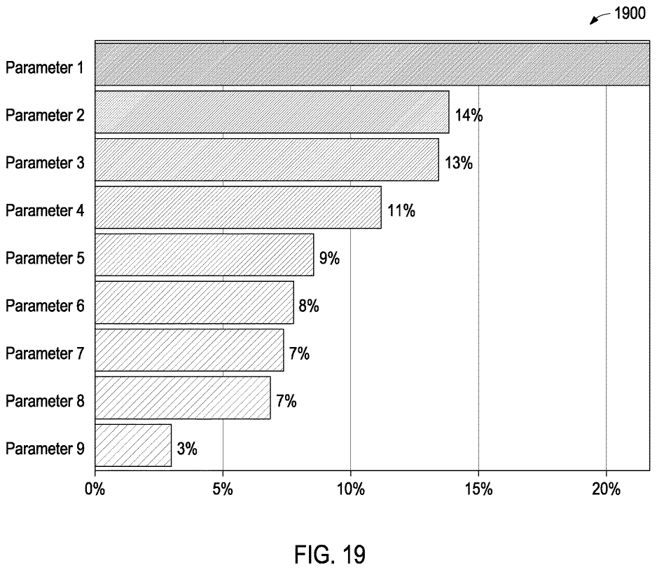

Furthermore, the controller 102 may obtain a status, or quality, of a seal applied to a vessel from the RF surgical instrument 106 using a machine learning model and any number of the parameters described elsewhere herein. For example, with reference now to FIG. 19, the controller 102 may use a machine learning model 1900 that includes nine inputs (Parameters 1-9) weighted based on their importance to the task of predicting seal quality. The weights provided in FIG. 19 are merely exemplary and may be adjusted as desired. Furthermore, the machine learning model may use less than nine inputs (e.g., 2, 3, 4, 5, 6, 7, or 8 inputs) or more than nine inputs (e.g., 10, 11, 12, 13, 14, or 15 inputs). The Parameters 1-9 may include any of the parameters described elsewhere herein and/or that are measurable by the various sensors described herein.

The machine learning model may include a Random Forest model with 100 trees, a max depth of 8, and a minimum sample per leaf of 4. However, the Random Forest model may use more or less than 100 trees, a max depth greater or less than 8, and a minimum sample per leaf greater or less than 4. The machine learning model is not limited to a Random Forest, but may be composed of any machine learning model including, but not limited to Logistic Regression, LightGMB, XGBoost, KNearest Neighbor, Neural Network, SVM, or Linear Regression, or combinations thereof.

Embodiments disclosed herein include:
A. A surgical system comprising a surgical instrument comprising an end effector that includes A first clamp arm, a second clamp arm movable relative to the first clamp arm from an open configuration to a closed configuration to engage a vessel therebetween, and an electrode, and a controller in operable communication with the surgical instrument, wherein the controller is operable to apply, with the electrode, energy to the vessel, estimate a time of media fracture of the vessel, measure an amount of the energy applied to the vessel after the estimated time, and estimate a quality of a seal applied to the vessel based on the measured amount of energy.
B. A surgical system comprising a surgical instrument comprising an end effector that includes a first clamp arm, a second clamp arm movable relative to the first clamp arm between an open configuration and a closed configuration to capture a vessel therebetween, and an electrode, a sensor to sense a position of the second clamp arm relative to the first clamp arm, and a controller in operable communication with the surgical instrument and the sensor, wherein the controller is operable to apply, with the electrode, energy to the vessel, monitor, with the sensor, a rate of motion and a position of the second clamp arm, and estimate a time of media fracture of the vessel based on a comparison of the rate of motion to a rate of motion threshold and detecting, or failing to detect, the second clamp arm reaching a threshold position.
C. A surgical system comprising a surgical instrument comprising an end effector that includes a first clamp arm, a second clamp arm movable relative to the first clamp arm between an open configuration and a closed configuration to capture a vessel therebetween, and an electrode, a sensor to sense a position of the second clamp arm relative to the first clamp arm, and a controller in operable communication with the surgical instrument and the sensor, wherein the controller is operable to apply, with the electrode, energy to the vessel, monitor, with the sensor, a rate of motion and a position of the second clamp arm, and estimate a quality of a seal applied to the vessel based on the rate of motion and the position.

Each of the embodiments A, B, and C may have one or more of the following additional elements in any combination: Element 1: further comprising a sensor to sense a position of the second clamp arm relative to the first clamp arm, the controller being further operable to monitor, via the sensor, a rate of motion of the second clamp arm and estimate the quality of the seal applied to the vessel based further on the monitored rate of motion. Element 2: wherein the controller is further operable to compare the monitored rate of motion to a rate of motion threshold and provide an alert based on the monitored rate of motion reaching or exceeding the rate of motion threshold. Element 3: further comprising a sensor to sense a position of the second clamp arm relative to the first clamp arm, and wherein to estimate the time, the controller is operable to monitor, with the sensor, the position of the second clamp arm, compare the monitored position to a threshold position, and detect, with the sensor, the second clamp arm reaching the threshold position, wherein a time of the second clamp arm reaching the threshold position corresponds to the estimated time. Element 4: wherein to estimate the time, the controller is further operable to fail to detect, with the sensor, the second clamp arm reaching the threshold position and determine a time of the rate of motion being at a maximum based on the controller failing to detect the second clamp arm reaching the threshold position, wherein the time of the rate of motion being at the maximum corresponds to the estimated time. Element 5: further comprising a temperature sensor for sensing a temperature of the vessel, and wherein the amount of energy applied to the vessel after the estimated time includes first energy applied to the vessel with the temperature below a threshold temperature and second energy applied to the vessel with the temperature above the threshold temperature, wherein the controller is operable to estimate the quality of the seal based on the first energy. Element 6: wherein the threshold temperature is about 100° C. (212° F.). Element 7: wherein the controller is operable to estimate the quality of the seal applied to the vessel using a machine learning model trained with a plurality of parameters including the measured amount of energy. Element 8: wherein the machine learning model is one of a Random Forest model, a Logistic Regression model, a LightGBM model, an XGBoost model, a KNearest Neighbor model, a Neural Network model, a linear regression, or an SVM model, or combinations thereof. Element 9: wherein, based on the controller detecting the second clamp arm reaching the threshold position, the controller is operable to estimate the time of media fracture as a time of the second clamp arm reaching the threshold position. Element 10: wherein, based on the controller failing to detect the second clamp arm reaching the threshold position, the controller is operable to estimate the time of media fracture as a time of the rate of motion being at a maximum. Element 11: wherein, based on the controller failing to detect the second clamp arm reaching the threshold position, the controller is operable to estimate the time of media fracture as a time of the rate of motion being at or below the rate of motion threshold for an elapsed predefined length of time. Element 12: wherein the controller is operable to provide an alert based on the rate of motion reaching or exceeding the rate of motion threshold. Element 13: wherein the controller is further operable to measure an amount of the energy applied to the vessel after the estimated time and estimate a quality of a seal applied to the vessel based on the measured amount of energy. Element 14: wherein the controller is further operable to detect, with the sensor, a maximum rate of motion of the second clamp arm and adjust the amount of the energy applied to the vessel after the estimated time based on the maximum rate of motion. Element 15: wherein the controller is further operable to detect, with the sensor, an initial position of the second clamp arm and adjust the amount of the energy applied to the vessel after the estimated time further based on the initial position. Element 16: further comprising a temperature sensor for sensing a temperature of the vessel, and wherein the amount of energy applied to the vessel after the estimated time includes first energy applied to the vessel with the temperature below a threshold temperature and second energy applied to the vessel with the temperature above the threshold temperature, wherein the controller is operable to estimate the quality of the seal based on the first energy. Element 17: wherein the threshold temperature is about 100° C. (212° F.). Element 18: wherein the controller is operable to sense a phase of a signal provided to the electrode, and wherein the amount of energy applied to the vessel after the estimated time includes first energy applied to the vessel with the phase below a phase threshold and second energy applied to the vessel with the phase above the phase threshold, wherein the controller is operable to estimate the quality of the seal based on the first energy. Element 19: wherein the controller is operable to sense an impedance of the vessel, and wherein the amount of energy applied to the vessel after the estimated time includes first energy applied to the vessel with the impedance below an impedance threshold and second energy applied to the vessel with the impedance above the impedance threshold, wherein the controller is operable to estimate the quality of the seal based on the first energy. Element 20: wherein the controller is operable to estimate the quality of the seal applied to the vessel using a machine learning model trained with a plurality of parameters including the measured amount of energy. Element 21: wherein the machine learning model is one of a Random Forest model, a Logistic Regression model, a LightGBM model, an XGBoost model, a KNearest Neighbor model, a Neural Network model, a linear regression, or an SVM model, or combinations thereof.

By way of non-limiting example, exemplary combinations applicable to A, B, and C include: Element 1 and Element 2; Element 3 and Element 4; Element 5 and Element 6; Element 7 and Element 8; Element 13 and Element 14; Element 13 and Elements 14 and 15; Element 13 and Elements 14 and 16; Element 14 and Elements 14, 16, and 17; Element 13 and Element 18; Element 13 and Element 19; Element 13 and Element 20; Element 13 and Elements 20 and 21.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A surgical system, comprising:
a surgical instrument comprising an end effector that includes:
a first clamp arm;
a second clamp arm movable relative to the first clamp arm from an open configuration to a closed configuration to engage a vessel therebetween; and
an electrode; and
a controller in operable communication with the surgical instrument, wherein the controller is operable to:
apply, with the electrode, energy to the vessel;
estimate a time of media fracture of the vessel;
measure an amount of the energy applied to the vessel after the estimated time; and
estimate a quality of a seal applied to the vessel based on the measured amount of the energy.

2. The surgical system of claim 1, further comprising a sensor to sense a position of the second clamp arm relative to the first clamp arm, the controller being further operable to:
monitor, via the sensor, a rate of motion of the second clamp arm; and
estimate the quality of the seal applied to the vessel based further on the monitored rate of motion.

3. The surgical system of claim 2, wherein the controller is further operable to:
compare the monitored rate of motion to a rate of motion threshold; and
provide an alert based on the monitored rate of motion reaching or exceeding the rate of motion threshold.

4. The surgical system of claim 1, further comprising a sensor to sense a position of the second clamp arm relative to the first clamp arm, and wherein to estimate the time, the controller is operable to:
monitor, with the sensor, the position of the second clamp arm;
compare the monitored position to a threshold position; and
detect, with the sensor, the second clamp arm reaching the threshold position,
wherein a time of the second clamp arm reaching the threshold position corresponds to the estimated time.

5. The surgical system of claim 4, wherein to estimate the time, the controller is further operable to:
fail to detect, with the sensor, the second clamp arm reaching the threshold position; and
determine a time of the rate of motion being at a maximum based on the controller failing to detect the second clamp arm reaching the threshold position,
wherein the time of the rate of motion being at the maximum corresponds to the estimated time.

6. The surgical system of claim 1, further comprising a temperature sensor for sensing a temperature of the vessel, and wherein the amount of the energy applied to the vessel after the estimated time includes:
first energy applied to the vessel with the temperature below a threshold temperature; and
second energy applied to the vessel with the temperature above the threshold temperature,
wherein the controller is operable to estimate the quality of the seal based on the first energy.

7. The surgical system of claim 1, wherein the controller is operable to sense a phase of a signal provided to the electrode, and wherein the amount of the energy applied to the vessel after the estimated time includes:
first energy applied to the vessel with the phase below a phase threshold; and
second energy applied to the vessel with the phase above the phase threshold,
wherein the controller is operable to estimate the quality of the seal based on the first energy or the second energy or a combination thereof.

8. The surgical system of claim 1, wherein the controller is operable to sense an impedance of the vessel, and wherein the amount of the energy applied to the vessel after the estimated time includes:
first energy applied to the vessel with the impedance below an impedance threshold; and
second energy applied to the vessel with the impedance above the impedance threshold,
wherein the controller is operable to estimate the quality of the seal based on the first energy or the second energy or a combination thereof.

9. The surgical system of claim 1, wherein the controller is operable to estimate the quality of the seal applied to the vessel using a machine learning model trained with a plurality of parameters including the measured amount of the energy.

10. The surgical system of claim 1, wherein the amount of energy is a first amount of energy and the controller is further operable to:
measure a second amount of the energy applied to the vessel prior the estimated time; and
estimate the quality of the seal applied to the vessel further based on the measured second amount of the energy.

11. A surgical system, comprising:
a surgical instrument comprising an end effector that includes:
a first clamp arm;
a second clamp arm movable relative to the first clamp arm between an open configuration and a closed configuration to capture a vessel therebetween; and
an electrode;
a sensor to sense a position of the second clamp arm relative to the first clamp arm; and
a controller in operable communication with the surgical instrument and the sensor, wherein the controller is operable to:
apply, with the electrode, energy to the vessel;
monitor, with the sensor, a rate of motion and a position of the second clamp arm; and
estimate a time of media fracture of the vessel based on:
a comparison of the rate of motion to a rate of motion threshold; and
detecting, or failing to detect, the second clamp arm reaching a threshold position.

12. The surgical system of claim 11, wherein, based on the controller detecting the second clamp arm reaching the threshold position, the controller is operable to estimate the time of media fracture as a time of the second clamp arm reaching the threshold position.

13. The surgical system of claim 11, wherein, based on the controller failing to detect the second clamp arm reaching the threshold position, the controller is operable to estimate the time of media fracture as a time of the rate of motion being at a maximum.

14. The surgical system of claim 11, wherein, based on the controller failing to detect the second clamp arm reaching the threshold position, the controller is operable to estimate the time of media fracture as a time of the rate of motion being at or below the rate of motion threshold for an elapsed predefined length of time.

15. The surgical system of clamp 11, wherein the controller is further operable to:
measure an amount of the energy applied to the vessel after the estimated time; and
estimate a quality of a seal applied to the vessel based on the measured amount of the energy.

16. The surgical system of claim 15, wherein the controller is further operable to:
detect, with the sensor, a maximum rate of motion of the second clamp arm; and
adjust the amount of the energy applied to the vessel after the estimated time based on the maximum rate of motion.

17. The surgical system of claim 16, wherein the controller is further operable to:
detect, with the sensor, an initial position of the second clamp arm; and
adjust the amount of the energy applied to the vessel after the estimated time further based on the initial position.

18. The surgical system of claim 15, further comprising a temperature sensor for sensing a temperature of the vessel, and wherein the amount of the energy applied to the vessel after the estimated time includes:
first energy applied to the vessel with the temperature below a threshold temperature; and
second energy applied to the vessel with the temperature above the threshold temperature,
wherein the controller is operable to estimate the quality of the seal based on the first energy.

19. The surgical system of claim 15, wherein the controller is operable to sense a phase of a signal provided to the electrode, and wherein the amount of the energy applied to the vessel after the estimated time includes:
first energy applied to the vessel with the phase below a phase threshold; and
second energy applied to the vessel with the phase above the phase threshold,
wherein the controller is operable to estimate the quality of the seal based on the first energy or the second energy or a combination thereof.

20. The surgical system of claim 15, wherein the controller is operable to sense an impedance of the vessel, and wherein the amount of the energy applied to the vessel after the estimated time includes:
first energy applied to the vessel with the impedance below an impedance threshold; and
second energy applied to the vessel with the impedance above the impedance threshold,
wherein the controller is operable to estimate the quality of the seal based on the first energy or the second energy or a combination thereof.

21. The surgical system of claim 15, wherein the controller is operable to estimate the quality of the seal applied to the vessel using a machine learning model trained with a plurality of parameters including the measured amount of the energy.

22. A surgical system, comprising:
a surgical instrument comprising an end effector that includes:
a first clamp arm;
a second clamp arm movable relative to the first clamp arm between an open configuration and a closed configuration to capture a vessel therebetween; and
an electrode;
a sensor to sense a position of the second clamp arm relative to the first clamp arm; and
a controller in operable communication with the surgical instrument and the sensor, wherein the controller is operable to:
apply, with the electrode, energy to the vessel;
monitor, with the sensor, a rate of motion and a position of the second clamp arm; and
estimate a quality of a seal applied to the vessel based on the rate of motion and the position.
